(19) United States
(12) Patent Application Publication
(10) Pub. No.: US 2010/0010025 A1
(43) Pub. Date: Jan. 14, 2010

(54) Pyrimidine Derivatives

(75) Inventors: Rudolf Duthaler, Basel (CH); Marc Gerspacher, Basel (CH); Philipp Holzer, Suhr (CH); Markus Streiff, Birsfelden (CH); Gebhard Thoma, Lorrach (DE); Rudolf Walchli, Basel (CH); Hans-Günter Zerwes, Lorrach (DE)

Correspondence Address: NOVARTIS INSTITUTES FOR BIOMEDICAL RESEARCH, INC., 220 MASSACHUSETTS AVENUE, CAMBRIDGE, MA, 02139, US

(73) Assignee: Norvartis AG

(21) Appl. No.: 12/374,524

(22) PCT Filed: Jul. 19, 2007
(86) PCT No.: PCT/EP07/06452

§ 371 Date: Jan. 21, 2009

(30) Foreign Application Priority Data
Jul. 21, 2006 (EP) 06117632.7

(51) Int. Cl. A61K 31/506; C07D 239/42; A61K 31/505; C07D 401/12; C07D 401/14

(52) U.S. Cl. 514/275; 544/332; 544/331

(57) ABSTRACT

Disclosed are pyrimidine derivatives having interesting pharmacological properties.

The present invention relates to pyrimidine derivatives, processes for their production, their use as pharmaceuticals and to pharmaceutical compositions comprising them.

More particularly the present invention provides in a first aspect a compound of formula I

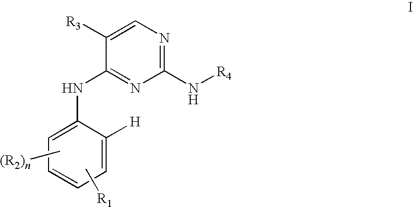

wherein
R₁, and R₂ are independently selected from H; X—SOₘ—Y wherein X is a direct bond, C₁₋₃alkylene, O or NRₐ wherein Rₐ is H or C₁₋₄alkyl; and Y is C₁₋₄alkyl or NR₁₁R₁₂ wherein each of R₁₁ and R₁₂, independently, is H or C₁₋₄alkyl; halogen; OH; C₁₋₇alkyl optionally substituted by OH or C₁₋₆alkoxy; C₁₋₇halogenoalkyl; C₁₋₇alkoxy; C₁-C₇alkoxy substituted by cyano; C₁₋₆alkylthio; C₂₋₇alkenyl; C₂₋₇alkynyl; C₃₋₇cycloalkyl; C₃₋₇cycloalkenyl; heterocyclyl; heterocyclylC₁₋₃alkyl; optionally substituted phenyl-R_{b} wherein R_{b} is a direct bond or a bridging group comprising 1 to 4 carbon atoms among which one C atom may be replaced by O or NRₓ, Rₓ being H or C₁₋₃alkyl; optionally substituted heteroaryl-R_{c} wherein R_{c} has independently one of the significances given for R_{b}; heteroaryl N-oxide; or heteroaryl N-oxide C₁₋₃alkyl;
or 2 adjacent R₂ form an annulated 5-12 membered nonaromatic ring optionally containing up to 4 groups selected from CO, NR₁₀, O, S, SO or SO₂;
with the proviso that R₁, and R₂ are not both H;

### R₃ is COOH, CONH₂ or CSNH₂;

R₄, is aryl or heteroaryl, each being optionally substituted by 1 to 4 substitutents R₈ selected from halogen; OH; C₁-C₇alkyl optionally substituted by OH or C₁₋₆alkoxy; C₁-C₇alkoxy; C₁₋₇halogenoalkyl; C₂₋₇alkenyl; C₂₋₇alkynyl; C₃₋₇cycloalkyl; C₃₋₇cycloalkenyl; heterocyclyl; heterocyclylC₁₋₃alkyl; aryl; phenyl; phenyl substituted by C₁-C₇alkyl, C₁₋₆alkoxy, NH₂, NHR₉, NR₉R₉, halogen, C₁₋₃acyl; heteroaryl; C₁₋₃acyl-heteroaryl; heteroarylC₁₋₃alkyl; heteroaryl N-oxideC₀-C₃alkyl; CONH₂; CONHR₉; CONR₉R₉; OC(O)R₉; OC(O)OR₉; OC(O)NHR₉; OC(O)NR₉R₉; OSO₂R₉; COOH; COOR₉; COR₉; X₁COOR₉; CN; NO₂; NH₂; NHR₉; NR₉R₉; X₁R₉R₉; NHC(O)R₉; NR₉C(O)R₉; NHC(O)NHR₉; NHC(O)NH₂; NR₉C(O)NHR₉; NR₉C(O)NR₉R₉; NHC(O)OR₉; NR₉C(O)OR₉; NHSO₂R₉; N(SO₂R₉)₂; NR₉SO₂R₉; SR₉; S(O)R₉; SO₂R₉; or Si(CH₃)₃;
or 2 adjacent R₈ form an annulated 5-12 membered nonaromatic ring optionally containing up to 4 groups selected from CO, CHCOOH, CHCOOR₉, NR₁₀, O, S, SO or SO₂;
each of R₉, independently, is C₁₋₆alkyl; C₂₋₆alkenyl; C₂₋₆alkynyl; C₂₋₄hydroxyalkyl; R₁₀O—C₂₋₄alkyl; R₁₀R₁₀N—C₂₋₄alkyl; C₃₋₆cycloalkyl; C₃₋₆cycloalkylC₁₋₃alkyl; phenyl; phenylC₁₋₃alkyl; heteroaryl; heteroarylC₁₋₃alkyl; heterocyclyl; heterocyclylC₁₋₃alkyl;
or 2 R₉ form together with the N atom to which they are attached, a 4 to 7 membered non-aromatic ring optionally containing up to 3 groups selected from CO, NR₁₀, O, S, SO or SO₂;
each of R₁₀, independently, is H; C₁₋₆alkyl; C₂₋₄hydroxyalkyl; or C₃₋₆cycloalkyl; or 2 R₁₀ form together with the N atom to which they are attached, a 4 to 7 membered non-aromatic ring; and
n is 1 or 2;
m is 1 or 2, preferably 2;
X₁ is a direct bond or C₁₋₆alkylene;
in free form or in salt form.

The present invention further relates to a compound of above formula I, wherein

R₁ is H; X—SOₘ—Y wherein X is a direct bond, C₁₋₃alkylene, O or NRₐ wherein Rₐ is H or C₁₋₄alkyl; and Y is C₁₋₄alkyl or NR₁₁R₁₂ wherein each of R₁₁ and R₁₂, independently, is H or C₁₋₄alkyl;
R₂ is H; halogen; OH; C₁₋₇alkyl optionally substituted by OH or C₁₋₆alkoxy; C₁₋₇halogenoalkyl; C₁₋₇alkoxy; C₁-C₇alkoxy substituted by cyano; C₁₋₆alkylthio; C₂₋₇alkenyl; C₂₋₇alkynyl; C₃₋₇cycloalkyl; C₃₋₇cycloalkenyl; heterocyclyl; heterocyclylC₁₋₃alkyl; optionally substituted phenyl-R_{b} wherein R_{b} is a direct bond or a bridging group comprising 1 to 4 carbon atoms among which one C atom may be replaced by O or NRₓ, Rₓ being H or C₁₋₃alkyl; optionally substituted heteroaryl-R_{c} wherein R_{c} has independently one of the significances given for R_{b}; heteroaryl N-oxide; or heteroaryl N-oxide C₁₋₃alkyl;
or 2 adjacent R₂ form an annulated 5-12 membered nonaromatic ring optionally containing up to 4 groups selected from CO, NR₁₀, O, S, SO or SO₂; with the proviso that and R₂ are not both H;

### R₃ is COOH, CONH₂ or CSNH₂;

R₄, is aryl or heteroaryl, each being optionally substituted by 1 to 4 substitutents R₈ selected from halogen; OH; C₁-C₇alkyl optionally substituted by OH or C₁₋₆alkoxy; C₁-C₇alkoxy; C₁₋₇halogenoalkyl; C₂₋₇alkenyl; C₂₋₇alkynyl; C₃₋₇cycloalkyl; C₃₋₇cycloalkenyl; heterocyclyl; heterocyclylC₁₋₃alkyl; aryl; phenyl; phenyl substituted by C₁-C₇alkyl, C₁₋₆alkoxy, NH₂, NHR₉, NR₉R₉, halogen, C₁₋₃acyl; heteroaryl; C₁₋₃acyl-heteroaryl; heteroarylC₁₋₃alkyl; heteroaryl N-oxideC₀-C₃alkyl; CONH₂; CONHR₉; CONR₉R₉; OC(O)R₉; OC(O)OR₉; OC(O)NHR₉; OC(O)NR₉R₉; OSO₂R₉; COOH; COOR₉; COR₉; X₁COOR₉; CN; NO₂; NH₂; NHR₉; NR₉R₉; X₁NR₉R₉; NHC(O)R₉; NR₉C(O)R₉; NHC(O)NHR₉; NHC(O)NH₂; NR₉C(O)NHR₉; NR₉C(O)NR₉R₉; NHC(O)OR₉; NR₉C(O)OR₉; NHSO₂R₉; N(SO₂R₉)₂; NR₉SO₂R₉; SR₉; S(O)R₉; SO₂R₉; or Si(CH₃)₃;
or 2 adjacent R₈ form an annulated 5-12 membered nonaromatic ring optionally containing up to 4 groups selected from CO, CHCOOH, CHCOOR₉, NR₁₀, O, S, SO or SO₂;
each of R₉, independently, is Cl_{—}6alkyl; C₂₋₆alkenyl; C₂₋₆alkynyl; C₂₋₄hydroxyalkyl;
R₁₀O—C₂₋₄alkyl; R₁₀R₁₀N—C₂₋₄alkyl; C₃₋₆cycloalkyl; C₃₋₆cycloalkylC₁₋₃alkyl; phenyl; phenylC₁₋₃alkyl; heteroaryl; heteroarylC₁₋₄alkyl; heterocyclyl; heterocyclylC₁₋₄alkyl; or 2 R₉ form together with the N atom to which they are attached, a 4 to 7 membered non-aromatic ring optionally containing up to 3 groups selected from CO, NR₁₀, O, S, SO or SO₂;
each of R₁₀, independently, is H; C₁₋₆alkyl; C₂₋₄hydroxyalkyl; or C₃₋₆cycloalkyl;
or 2 R₁₀ form together with the N atom to which they are attached, a 4 to 7 membered non-aromatic ring; and
n is 1 or 2;
m is 1 or 2, preferably 2;
X₁ is a direct bond or C₁₋₄alkylene;
in free form or in salt form.

As indicated above, whenever R₁ and R₂ can stand for hydrogen, at least one of R₁ or R₂must not be hydrogen.

Preferably n is 1.

Preferably, R₁ and R₂ shall not both stand for X—SOₘ—Y.

In a preferred embodiment R₁ is X—SOₘ—Y and R₂ is hydrogen.

Preferably R₁ is X—SOₘ—Y wherein X is a direct bond, C₁₋₃alkylene, O or NRₐ wherein Rₐ is H or C₁₋₄alkyl; and Y is C₁₋₄alkyl or NR₁₁R₁₂ wherein each of R₁₁ and R₁₂, independently, is H or C₁₋₄alkyl; and wherein m is 1 or 2, preferably 2.

Preferably Y is C₁₋₄alkyl, in particular methyl, ethyl, n-propyl, i-propyl, n butyl, sec-butyl, tert-butyl, or iso-butyl, more preferably methyl.

Preferably, R₁ is H; and R₂ is halogen; OH; C₁₋₇alkyl optionally substituted by OH or C₁₋₆alkoxy; Cl_{—}7halogenoalkyl; C₁₋₇alkoxy; C₁-C₇alkoxy substituted by cyano; C₁₋₆alkylthio; C₂₋₇alkenyl; C₂₋₇alkynyl; C₃₋₇cycloalkyl; C₃₋₇cycloalkenyl; heterocyclyl; heterocyclylC₁₋₃alkyl; optionally substituted phenyl-R_{b} wherein R_{b} is a direct bond or a bridging group comprising 1 to 4 carbon atoms among which one C atom may be replaced by O or NRₓ, Rₓ being H or C₁₋₃alkyl; optionally substituted heteroaryl-R_{c} wherein R_{c} has independently one of the significances given for R_{b}; heteroaryl N-oxide; or heteroaryl N-oxide C₁₋₃alkyl;

or 2 adjacent R₂ form an annulated 5-12 membered nonaromatic ring optionally containing up to 4 groups selected from CO, NR₁₀, O, S, SO or SO₂ and
n is 1 or 2;
also preferably R₁ is H; and R₂ is halogen; OH; C₁₋₇alkyl optionally substituted by OH or C₁₋₆alkoxy; C₁₋₇halogenoalkyl; C₁₋₇alkoxy; C₁-C₇alkoxy substituted by cyano; or C₁₋₆alkylthio; and n is 1 or 2.

Preferably, R₃ is CONH₂ and R₄ is aryl being optionally substituted by 1 to 4 substitutents R₈ selected from halogen; OH; C₁-C₇alkyl optionally substituted by OH or C₁₋₆alkoxy; C₁-C₇alkoxy; C₁₋₇halogenoalkyl; C₂₋₇alkenyl; C₂₋₇alkynyl; C₃₋₇cycloalkyl; C₃₋₇cycloalkenyl; heterocyclyl; heterocyclylC₁₋₃alkyl; phenyl; phenyl substituted by C₁-C₇alkyl, C₁₋₆alkoxy, NH₂, NHR₉, NR₉R₉, halogen, C₁₋₃acyl; phenyl substituted by 1-3 halogen; phenyl substituted by 1-3 carbamoyl; heteroaryl; d-3acyl-heteroaryl; heteroarylC₁₋₃alkyl; heteroaryl N-oxideC₀-C₃alkyl; CONH₂; CONHR₉; CONR₉R₉; OC(O)R₉; OC(O)OR₉; OC(O)NHR₉; OC(O)NR₉R₉; OSO₂R₉; COOH; COOR₉; COR₉; X₁COOR₉; CN; NO₂; NH₂; NHR₉; NR₉R₉; X₁NR₉R₉; NHC(O)R₉; NR₉C(O)R₉; NHC(O)NHR₉; NHC(O)NH₂; NR₉C(O)NHR₉; NR₉C(O)NR₉R₉; NHC(O)OR₉; NR₉C(O)OR₉; NHSO₂R₉; N(SO₂R₉)₂; NR₉SO₂R₉; SR₉; S(O)R₉; SO₂R₉; or Si(CH₃)₃;

or 2 adjacent R₈ form an annulated 5-12 membered nonaromatic ring optionally containing up to 4 groups selected from CO, CHCOOH, CHCOOR₉, NR₁₀, O, S, SO or SO₂;
each of R₉, independently, is C₁₋₆alkyl; C₂₋₆alkenyl; C₂₋₆alkynyl; C₂₋₄hydroxyalkyl; R₁₀O—C₂₋₄alkyl; R₁₀R₁₀N—C₂₋₄alkyl; C₃₋₆cycloalkyl; C₃₋₆cycloalkylC₁₋₃alkyl; phenyl; phenylC₁₋₃alkyl; heteroaryl; heteroarylC₁₋₃alkyl; heterocyclyl; heterocycrylC₁₋₃alkyl;
or 2 R₉ form together with the N atom to which they are attached, a 4 to 7 membered non-aromatic ring optionally containing up to 3 groups selected from CO, NR₁₀, O, S, SO or SO₂;
each of R₁₀, independently, is H; C₁₋₆alkyl; C₂₋₄hydroxyalkyl; or C₃₋₆cycloalkyl; or 2 R₁₀ form together with the N atom to which they are attached, a 4 to 7 membered non-aromatic ring; and
n is 1 or 2.

Preferably, R₃ is CONH₂ and R₄ is a radical of formula Ia wherein the free valence (atom to which it is attached) is indicated by the free bond

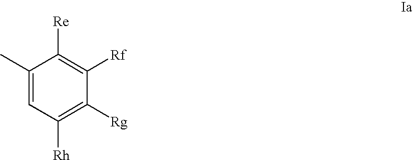

wherein Rₑ is H, Hal, or amino;
R_{f} is H or C₁₋₆alkoxy;
R_{g} is H, C₁₋₆alkoxy, CONHR₉ or CONR₉R₉; and
Rₕ is selected from halogen; C₁-C₇alkyl; C₁₋₆alkoxy; C₁₋₇halogenoalkyl; C₃₋₇cycloalkyl; heterocyclyl; phenyl; phenyl substituted by C₁-C₇alkyl, C₁₋₆alkoxy, NH₂, NHR₉, NR₉R₉, halogen, C₁₋₃acyl; carbamoylphenyl; heteroaryl; C₁₋₃acyl-heteroaryl; CONH₂; CONHR₉; CONR₉R₉; OC(O)R₉; COOH; COOR₉; COR₉; X₁COOR₉; CN; NO₂; NH₂; NHR₉; NR₉R₉; X₁NR₉R₉; NHC(O)R₉; NR₉C(O)R₉; NHC(O)NHR₉; NHC(O)NH₂; NR₉C(O)NHR₉; NR₉C(O)NR₉R₉; NHC(O)OR₉; and NR₉C(O)OR₉;
or R_{g} and Rₕ form an annulated 5-12 membered nonaromatic ring optionally containing up to 4 groups selected from CO, CHCOOH, CHCOOR₉, NR₁₀, O, S, SO or SO₂;
wherein R₉, R₁₀, and X₁ are as defined above.

In a preferred embodiment R₁ is H, R₃ is CONH₂ and R₄ is a radical of formula Ia, in which Rₕ is selected from C₁-C₇alkyl; C₁₋₆alkoxy; C₁₋₇halogenoalkyl; C₃₋₇cycloalkyl; heterocyclyl; phenyl; phenyl substituted by C₁-C₇alkyl, C₁₋₆alkoxy, NH₂, NHR₉, NR₉R₉, halogen, C₁₋₃acyl; carbamoylphenyl; heteroaryl; C₁-C₇alkyl-heteroaryl and C₁₋₃acyl-heteroaryl and Rₑ, R_{f} and R_{g} are as described above.

Preferably, Rₑ is halogen or hydrogen, more preferably fluoro.

In another preference, R₂ is hydrogen.

Any alkyl or alkyl moiety may be linear or branched. Halogen may be F, Cl, Br, or I, preferably F.

Aryl may be phenyl or naphthyl, preferably phenyl. Heteroaryl may be a mono-, bi- or tricyclic aromatic system comprising 1 to 4 heteroatoms selected from N, O and S, e.g. furyl, thienyl, pyrrolyl, imidazolyl, pyrazolyl, thiazolyl, isothiazolyl, oxazolyl, isoxazolyl, oxadiazolyl, thiadiazolyl, triazolyl, tetrazolyl, pyridyl, pyridazinyl, pyrimidinyl, pyrazinyl, triazinyl, tetrazinyl, indolyl, benzothiophenyl, benzofuranyl, benzimidazolyl, indazolyl, benzotriazolyl, benzothiazolyl, benzoxazolyl, quinolinyl, isoquinolinyl, phthalazinyl, quinoxalinyl, quinazolinyl, cinnolinyl or naphthyridinyl.

Heterocyclyl is a 5, 6 or 7 membered non-aromatic heterocyclic ring which may be linked via C or N and may comprise 1, 2 or 3 groups selected e.g. from CO, NR₁₀, O, S, SO or SO₂. Examples are e.g. morpholinyl, piperazinyl, pyrrolidinyl, 2-oxopyrrolidinyl, 2,5-dioxopyrrolidinyl, or piperidyl. A 4 to 7 membered non-aromatic ring as formed by 2 R₉ or 2 R₁₀ groups together with the N to which they are attached, respectively, may be a 4 to 7 membered saturated or unsaturated heterocyclic ring which is linked via its N atom. Examples include e.g. piperidyl or pyrazolidinyl.

When R₂ is substituted phenyl-R_{b} or substituted heteroaryl-R_{c}, it is phenyl-R_{b} or heteroaryl-R_{c} which may have 1 to 3 substituents on the phenyl or heteroaryl ring and selected from halogen, C₁₋₄alkyl, C₁₋₄alkoxy, NR_{y}R_{y} and acyl. Each of R_{y}, independently, may be H, C₁₋₄alkyl or acyl.

Acyl may be a radical R_{d}CO wherein R_{d} is C₁₋₄alkyl, C₃₋₆cycloalkyl, phenyl or benzyl.

Examples of bridging group as R_{b} or R_{c} include e.g. C₁₋₄alkylene, —OC₁₋₄alkylene or —NHC₁₋₄alkylene.

X is preferably a direct bond or NRₐ.

X₁ is preferably CH₂.

R₃ is preferably CONH₂.

The compounds of formula I may exist in free form or in salt form, e.g. addition salts with e.g. organic or inorganic acids, for example trifluoroacetic or hydrochloride acid; or when R₃ is COOH, it may also be present in salt form, e.g. an ammonium salt or salts with metals such as sodium, potassium, calcium, zinc or magnesium; or a mixture thereof.

The present invention also provides a process for the production of a compound of formula I, comprising converting a compound of formula II

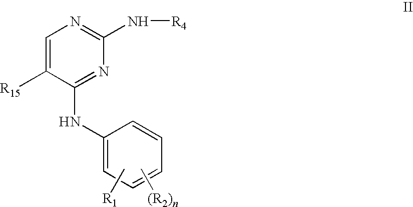

wherein n, R₁, R₂ and R₄ is as defined above, and R₁₅ is a group which can be converted to R₃, e.g. COOH or an ester group, e.g. COOR₁₃ wherein R₁₃ is C₁₋₆alkyl
and recovering the resulting compound of formula I in free or in form of a salt, and, where required, converting the compound of formula I obtained in free form into the desired salt form, or vice versa.

The process may be performed according to methods known in the art, e.g. as described in the examples hereinafter.

Compounds of formula II, used as starting materials, may be produced by reacting a compound of formula III

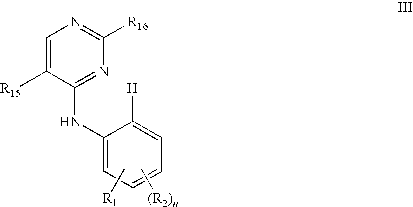

wherein n, R₁, R₂ and R₁₅ is as defined above, and R₁₆ is a leaving group, e.g. a halogen, e.g. F, Cl or Br, SR₁₄, SOR₁₄ or SO₂R₁₄ wherein R₁₄ is C₁₋₆alkyl
with a compound of formula IV

R₄—NH₂ IV

wherein R₄ is as defined above.

The reaction may be performed in accordance with methods known in the art or as disclosed hereinafter.

Compounds of formula III may be prepared by reacting a compound of formula V

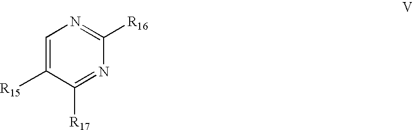

wherein R₁₅ and R₁₆ are as defined above and R₁₇ is, independently, a leaving group, e.g. a halogen, e.g. F, Cl or Br,
with a compound of formula VI

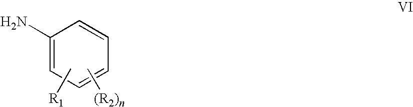

wherein R₁, R₂ and n are as defined above. The reaction may be carried out in accordance with methods known in the art or e.g. as disclosed thereafter.

Alternatively, a compound of formula II may be prepared by reacting a compound of formula VII,

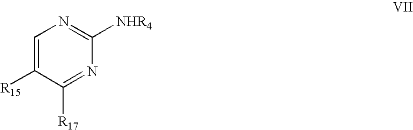

wherein R₄ and R₁₅ are as defined above, R₁₇ is a leaving group, e.g. Cl, F, or Br,
with a compound of formula VI optionally in the presence of a acid catalyst, or with a base to neutralize the acid formed.

Compounds of formula VII may be prepared from a compound of formula VIII,

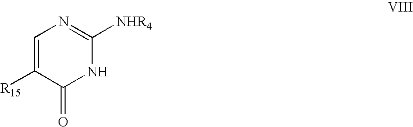

wherein R₄ and R₁₅ are as defined above. The conversion may be carried out in accordance with known methods.

Compounds of formulae V, VI, and VIII are either commercially available, known in the literature, or can be prepared by known methods.

Insofar as the production of the starting materials is not particularly described, the compounds are known or may be prepared analogously to methods known in the art or as disclosed in the Examples hereinafter.

The following examples illustrate the invention without any limitation.

The following abbreviations are employed:

Products were characterized by Ultra Performance Liquid Chromatography (HPLC, Acquity, Waters)-MS (ZQ, Waters) using a BEHC₁₈ column (1.7 jam, 2.1×50 mm). Method A: H₂O (0.1% formic acid)/CH₃CN, 0.7 mL/min, gradient: 80/20 to 10/90 in 4.2 min. Method B: H₂O (0.1% formic acid)/CH₃CN, 0.7 mL/min, gradient: 95/5 to 10/90 in 4.0 min. Method C: H₂O (0.1% formic acid)/CH₃CN, 0.7 mL/min, gradient: 99/1 to 1/99 in 2.25 min.
Ultra Performance Liquid Chromatography (HPLC, Acquity, Waters)-MS (ZQ, Waters) using a BEH SHIELD RP18 column (1.7 μm, 2.1×50 mm). Method D: H₂O (3 mM ammonium acetate+0.05% formic acid)/CH₃CN (0.05% formic acid), 0.5 mL/min, gradient: 98/2 to 2/98 in 5.0 min. at 50° C.
Liquid Chromatography (LC, Agilent 1100)-MS (ZQ 2000, Waters) using a Waters XTerra C₁₈ column (2.5 μm, 3×30 mm). Method E: Solvent A: H₂O, 5% CH₃CN (0.2% formic acid), Solvent B: CH₃CN (0.2% formic acid. Flow: 0.7-0.8 mL/min. Gradient: 0-2.5 min, A/B 5/95, 2.5-3 min, A/B 95/5, 50° C.
Liquid Chromatography (LC, Waters alliance 2690) Method F: gradient: water (0.1% TFA)/acetonitrile (0.1% TFA)= 98/2 for 1 min. to 100% acetonitrile (0.1% TFA) in 10 min. Stay at 100% for 2 min (total run time: 13 min.) Column: Column Engineering, Inc., Matrix, 3 μm C18 150×4.6 mm (Lot # 205) Detection by UV absorption (Waters Photodiode Array Detector 996) at 215 and 254 nm. The column temperature is 35° C. and the retention times are given in minutes. Flow rate: 1 mL/min.
Method G: Liquid chromatography (Cap LC Thermo Finnigan LTQ Sunfire) using a column (2.5 μM, 1×50 mm) H₂O (3 mM ammonium acetate/acetonitril+0.05% formic acid. Flow 35 μL/min

### EXAMPLE 1

### 2-(3,5-Dimethoxy-phenylamino)-4-(2-methanesulfonyl-phenylamino)-pyrimidine-5-carboxylic acid (1)

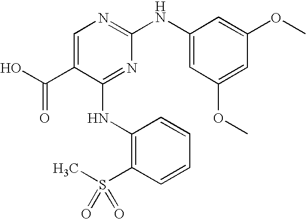

Step a: 2-(3,5-Dimethoxy-phenylamino)-6-oxo-1,6-dihydro-pyrimidine-5-carboxylic acid ethyl ester (1a)

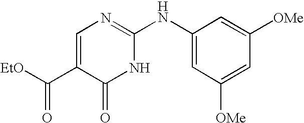

A solution of 2-methylsulfanyl-6-oxo-1,6-dihydro-pyrimidine-5-carboxylic acid ethyl ester (CA Reg. No. 53554-29-3, 300 mg) and 3,5-dimethoxy-phenylamine (CA Reg. No. 10272-08-8, 214 mg) in N,N-dimethylformamide (0.3 mL) is heated for 14 h to 130° C. The solvent is evaporated under reduced pressure, and the residue is crystallized from methanol, affording 1a (HPLC: method C, t^{ret} 1.79 min, MS 320/ES⁺).

Step b: 4-Chloro-2-(3,5-dimethoxy-phenylamino)-5-ethoxycarbonyl-pyrimidinium chloride (1b)

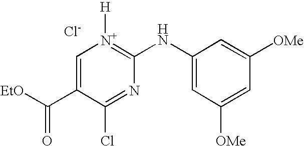

A solution of 1a (172 mg) in phosphoroxy-trichloride (3 mL) is heated for 2 h to 80° C. The reagent is evaporated at reduced pressure, the residue triturated with methanol and hexane. The precipitates (HPLC: method C, t^{ret} 2.22 min, 80%, MS 338/ES⁺) are directly used for the next step without purification.

Step c: 2-(3,5-Dimethoxy-phenylamino)-4-(2-methanesulfonyl-phenylamino)-pyrimidine-5-carboxylic acid ethyl ester (1c)

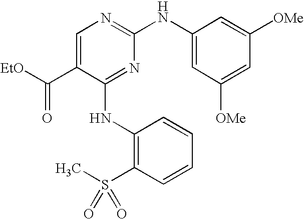

A solution of crude 1b (160 mg) and 2-methanesulfonyl-phenylammonium chloride (CA Reg. No. 2987-49-7, 98 mg) in 2-propanol (10 mL) and 4N hydrochloric acid (0.47 mL) is heated under reflux for 4 h. After removal of the solvents by evaporation, the residue is partitioned between ethyl acetate and aqueous ammonia. The dried organic phase is evaporated. The product is isolated from the residue by crystallization from ethyl acetate/hexane and chromatography of the mother liquors on silica gel (ethyl acetate/hexane 4:6). HPLC: method C, t^{ret} 2.19 min, MS 473/ES⁺.

Step d: 2-(3,5-Dimethoxy-phenylamino)-4-(2-methanesulfonyl-phenylamino)-pyrimidine-5-carboxylic acid (1)

A solution of 1c (50 mg) in 28% aqueous ammonia (12 mL) is heated for 16 h to 110° C. in an autoclave. The solvent is evaporated at reduced pressure, the residue acidified with 2 drops of concentrated (37%) hydrochloric acid. Repeated co-evaporation with dichloromethane affords 1, HPLC/MS: Method C, t^{ret} 3.09 Min, MS 445/ES⁺.

### EXAMPLE 2

### 2-(3,5-Dimethoxy-phenylamino)-4-(2-methanesulfonyl-phenylamino)-pyrimidine-5-carboxylic acid amide (2)

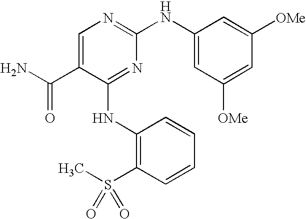

To a suspension of 2-(3,5-Dimethoxy-phenylamino)-4-(2-methanesulfonyl-phenylamino)-pyrimidine-5-carboxylic acid according to Example 1 (47 mg) in dichloromethane (8 mL) there is added para-N,N-dimethylamino-pyridine (52 mg), followed by ammonium chloride (56 mg) and (benzotriazol-1-yloxy)-tris-(dimethylamino)-phosphonium hexafluorophosphate (70 mg). After stirring for 30 min at room temperatures, the mixture is partitioned between water and ethyl acetate. The organic phase is washed with saturated brine, dried with Na₂SO₄, and evaporated. Chromatography of the residue (silica gel) eluting with ethyl acetate 10% methanol and precipitation with hexane yielded amide 2, HPLC/MS: method B, t^{ret} 3.04 min (89.6%), MS 444/ES⁺.

### EXAMPLE 3

### 2-(2-Fluoro-5-methoxy-phenylamino-4-(2-methanesulfonyl-phenylamino)-pyrimidine-5-carboxylic acid amide (3)

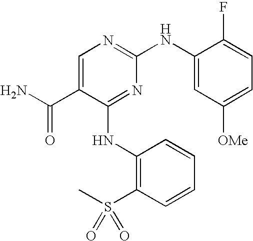

Step a: 2-(2-Fluoro-5-methoxy-phenylamino)-6-oxo-1,6-dihydro-pyrimidine-5-carboxylic acid ethyl ester (3a)

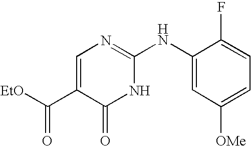

A mixture of 2-methylsulfonyl-6-oxo-1,6-dihydro-pyrimidine-5-carboxylic acid ethyl ester (CA Reg. No. 53554-29-3, 108 mg) and 2-fluoro-5-methoxy-aniline (CA Reg. No. 62257-15-2, 90 mg) is heated without solvent in an oil bath of 160° C. After 2 h the reaction is cooled, and the residue is crystallized from methanol affording 3a (UPLC: method C, t^{ret} 1.93 min, MS 308/ES⁺)

Step b: 4-Chloro-5-ethoxycarbonyl-2-(2-fluoro-5-methoxy-phenylamino)-pyrimidin-1-ium chloride/phosphate/chlorophosphates (3b)

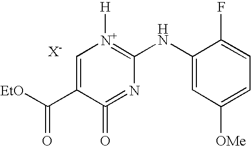

A solution of 3a (111 mg) in phosphoroxy-trichloride (3 mL) is heated for 45 min to 80° C. The reagent is evaporated at reduced pressure. The solid residue consisting of mixed salts 3b is used directly for step c (UPLC: method C, t^{ret} 2.22 min, MS 326, 328/ES⁺).

Step c: 2-(2-Fluoro-5-methoxy-phenylamino)-4-(2-methanesulfonyl-phenylamino)-pyrimidine-5-carboxylic acid ethyl ester (3c)

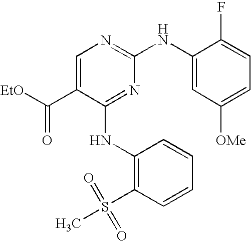

A solution of crude 3b (100 mg) and 2-methanesulfonyl-phenylammonium chloride (CA Reg. No. 2987-49-7, 64 mg) in 2-propanol (10 mL) is heated under reflux for 2.5 h. After removal of the solvents by evaporation, the residue is partitioned between ethyl acetate and aqueous ammonia. The organic phase is washed with saturated brine, dried (Na₂SO₄), and evaporated. Chromatography (silica gel, ethyl acetate/hexanes 54:45) and crystallization from ethyl acetate/hexanes affords 3c (UPLC: method C, t^{ret} 2.25 min, MS 461/ES⁺).

Step d: 2-(2-Fluoro-5-methoxy-phenylamino)-4-(2-methanesulfonyl-phenylamino)-pyrimidine-5-carboxylic acid amide (3)

A solution of 3c (32 mg) in condensed ammonia (3 mL) and methanol (2 mL) is heated in an autoclave to 50° C. After 48 h the vessel is cooled and ammonia and solvent evaporated. The residue is crystallized from ethyl acetate. Chromatography of the crystallizate (silica gel, ethyl acetate/methanol 96:4) affords 3 (UPLC: method C, t^{ret} 1.96 min, MS 432/ES⁺).

By following the procedure of Examples 1 to 3, the compounds disclosed in Table 1 are obtainable:

TABLE 1

| | | | UPLC | |
|---|---|---|---|---|
| | | | method | MS |
| Example | Formula | Name | (t^{ret}: min) | (ES⁺) |
| 4 | 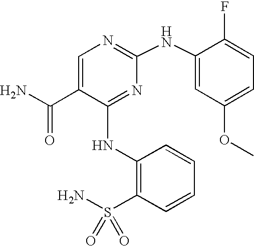 | 2-(2-Fluoro-5-methoxy- phenylamino)-4-(2-sulfamoyl- phenylamino)-pyrimidine-5- carboxylic acid amide | A 1.93 min | 433 |
| 5 | 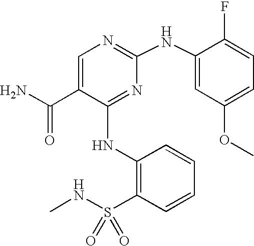 | 2-(2-Fluoro-5-methoxy- phenylamino)-4-(2- methylsulfamoyl- phenylamino)-pyrimidine-5- carboxylic acid amide | C 2.02 min | 447 |
| 6 | 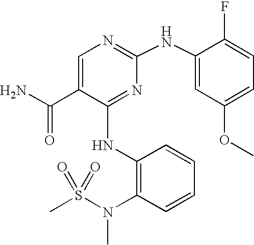 | 2-(2-Fluoro-5-methoxy- phenylamino)-4-[2- (methanesulfonyl-methyl- amino)-phenylamino]- pyrimidine-5-carboxylic acid amide | C 2.00 min | 461 |
| 7 | 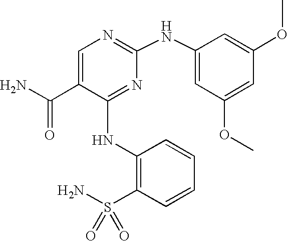 | 2-(3,5-Dimethoxy- phenylamino)-4-(2-sulfamoyl- phenylamino)-pyrimidine-5- carboxylic acid amide | C 1.89 min | 445 |
| 8 | 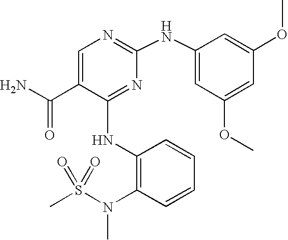 | 2-(3,5-Dimethoxy- phenylamino)-4-[2- (methanesulfonyl-methyl- amino)-phenylamino]- pyrimidine-5-carboxylic acid amide | C 1.94 min | 473 |
| 9 | 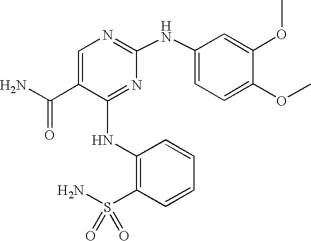 | 2-(3,4-Dimethoxy- phenylamino)-4-(2-sulfamoyl- phenylamino)-pyrimidine-5- carboxylic acid amide | A 0.91 min | 445 |
| 10 | 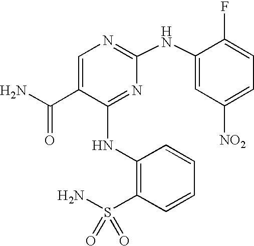 | 2-(2-Fluoro-5-nitro- phenylamino)-4-(2-sulfamoyl- phenylamino)-pyrimidine-5- carboxylic acid amide | C 1.92 | 448 |
| 11 | 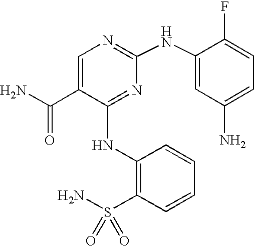 | 2-(5-Amino-2-fluoro- phenylamino)-4-(2-sulfamoyl- phenylamino)-pyrimidine-5- carboxylic acid amide | E 1.20 | 418 |
| 12 | 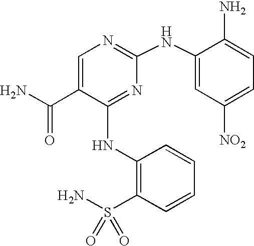 | 2-(2-Amino-5-nitro- phenylamino)-4-(2-sulfamoyl- phenylamino)-pyrimidine-5- carboxylic acid amide | C 1.80 | 445 |
| 13 | 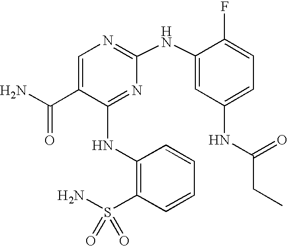 | 2-(2-Fluoro-5- propionylamino- phenylamino)-4-(2-sulfamoyl- phenylamino)-pyrimidine-5- carboxylic acid amide | E 1.35 | 474 |
| 14 | 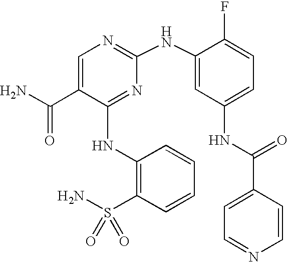 | 2-{2-Fluoro-5-[(pyridine-4- carbonyl)-amino]- phenylamino}-4-(2-sulfamoyl- phenylamino)-pyrimidine-5- carboxylic acid amide | C 1.73 | 523 |
| 15 | 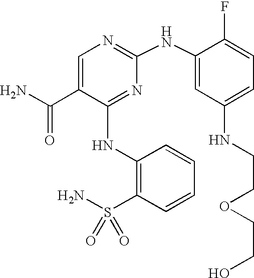 | 2-{2-fluoro-5-[2-(2-hydroxy- ethoxy)-ethylamino]- phenylamino}-4-(2-sulfamoyl- phenylamino)-pyrimidine-5- carboxylic acid amide | C 1.75 | 506 |
| 16 | 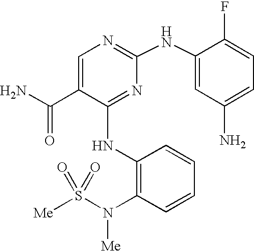 | 2-(5-Amino-2-fluoro- phenylamino)-4-[2- (methanesulfonyl-methyl- amino)-phenylamino]- pyrimidine-5-carboxylic acid amide | C 1.72 | 446 |
| 17 | 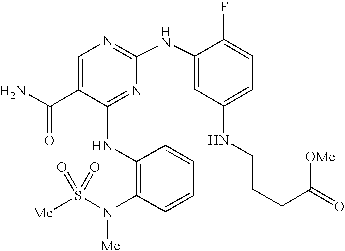 | 4-(3-{5-Carbamoyl-4-[2- (methanesulfonyl-methyl- amino)-phenylamino]- pyrimidin-2-ylamino}-4-fluoro- phenylamino)-butyric acid methyl ester | C 2.88 | 546 |
| 18 | 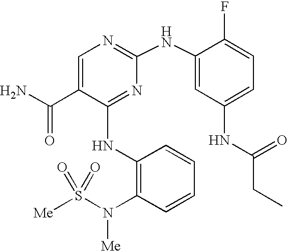 | 2-(2-Fluoro-5-propionyl- amino-phenylamino)-4-[2- (methanesulfonyl-methyl- amino)-phenylamino]- pyrimidine-5-carboxylic acid amide | C 1.81 | 502 |
| 19 | 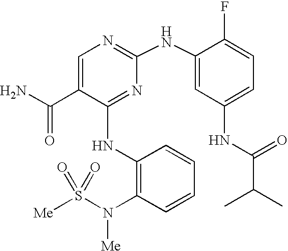 | 2-(2-Fluoro-5-isobutyryl- amino-phenylamino)-4-[2- (methanesulfonyl-methyl- amino)-phenylamino]- pyrimidine-5-carboxylic acid amide | C 1.88 | 516 |
| 20 | 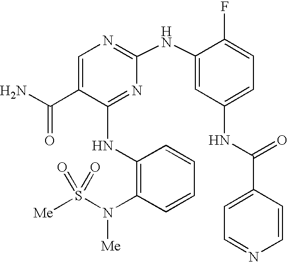 | 2-{2-Fluoro-5-[(pyridine-4- carbonyl)-amino]-phenyl- amino}-4-[2-(methane- sulfonyl-methyl-amino)-phenyl- amino]-pyrimidine-5- carboxylic acid amide | C 1.77 | 551 |
| 21 | 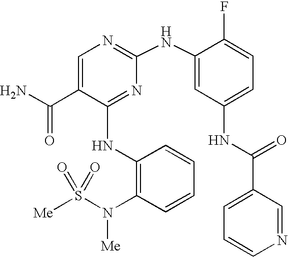 | 2-{2-Fluoro-5-[(pyridine-3- carbonyl)-amino]-phenyl- amino}-4-[2-(methane- sulfonyl-methyl-amino)-phenyl- amino]-pyrimidine-5- carboxylic acid amide | C 1.81 | 551 |
| 22 | 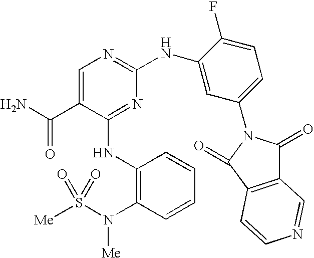 | 2-[5-(1,3-dioxo-1,3-dihydro- pyrrolo[3,4-c]pyridine-2-yl)-2- fluoro-phenylamino]-4-[2- (methanesulfonyl-methyl- amino)-phenylamino-pyrimi- dine-5-carboxylic acid amide | C 1.82 | 577 |
| 23 | 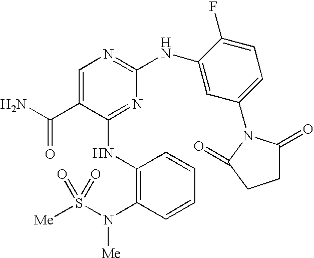 | 2-[5-(2,5-Dioxo-pyrrolidin-1- yl)-2-fluoro-phenylamino]-4- [2-methanesulfonyl-methyl- amino]-phenylamino]-pyrimi- dine-5-carboxylic acid amide | C 1.81 | 528 |
| 24 | 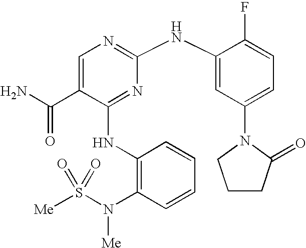 | 2-[2-Fluoro-5-(2-oxo-pyrroli- din-1-yl)-phenylamino]-4-[2- (methanesulfonyl-methyl- amino)-phenylamino]-pyrimi- dine-5-carboxylic acid amide | C 1.82 | 514 |
| 25 | 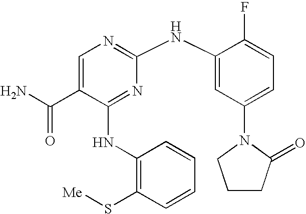 | 2-[2-Fluoro-5-(2-oxo-pyrroli- din-1-yl)-phenylamino]-4-(2- methylsulfanyl-phenylamino)- pyrimidine-5-carboxylic acid amide | C 1.92 | 453 |
| 26 | 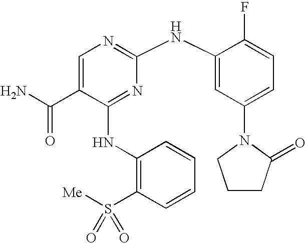 | 2-[2-Fluoro-5-(2-oxo-pyrroli- din-1-yl)-phenylamino]-4-(2- methylsulfonyl-phenylamino)- pyrimidine-5-carboxylic acid amide | C 1.83 | 485 |
| 27 | 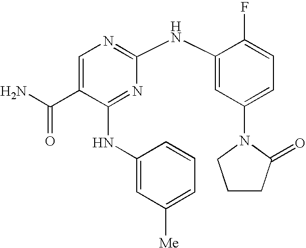 | 2-[2-Fluoro-5-(2-oxo-pyrroli- din-1-yl)-phenylamino]-4- m-tolylamino-pyrimidine-5- carboxylic acid amide | C 1.91 | 421 |
| 28 | 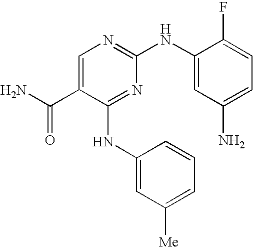 | 2-(5-Amino-2-fluoro- phenylamino)-4- m-tolylamino-pyrimidine-5- carboxylic acid amide | E 1.33 | 353 |
| 29 | 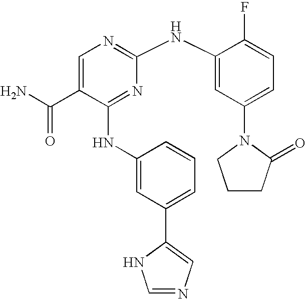 | 2-[2-Fluoro-5-(2-oxo-pyrroli- din-1-yl)-phenylamino]-4-[3- (3H-imidazol-4-yl)-phenylamino]- pyrimidine-5-carboxylic acid amide | C 1.64 | 473 |
| 30 | 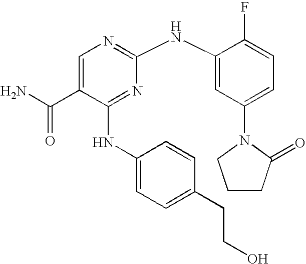 | 2-[2-Fluoro-5-(2-oxo-pyrroli- din-1-yl)-phenylamino]-4-[4- (2-hydroxy-ethyl)- phenylamino]-pyrimidine-5- carboxylic acid amide | C 1.72 | 451 |
| 31 | 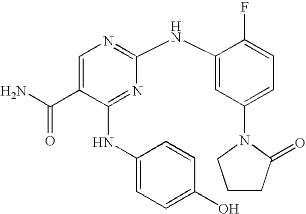 | 2-[2-Fluoro-5-(2-oxo-pyrroli- din-1-yl)-phenylamino]-4-(4- hydroxy-phenylamino)- pyrimidine-5-carboxylic acid amide | C 1.73 | 423 |
| 32 | 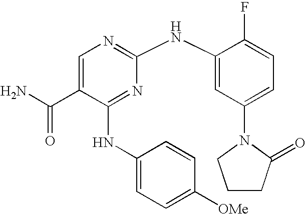 | 2-[2-Fluoro-5-(2-oxo-pyrroli- din-1-yl)-phenylamino]-4-(4- methoxy-phenylamino)- pyrimidine-5-carboxylic acid amide | C 1.86 | 437 |
| 33 | 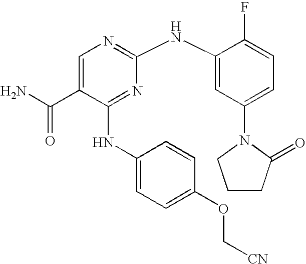 | 4-(4-cyanomethoxy- phenylamino)-2-[2-fluoro-5- (2-oxo-pyrrolidin-1-yl)- phenylamino]-pyrimidine-5- carboxylic acid amide | C 1.82 | 462 |
| 34 | 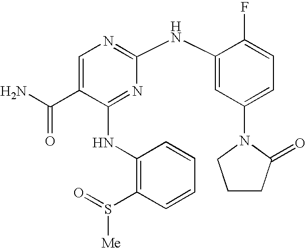 | 2-[2-Fluoro-5-(2-oxo- pyrrolidin-1-yl)-phenylamino]- 4-(2-methanesulfinyl- phenylamino)-pyrimidine-5- carboxylic acid amide | not det. | 469 |
| 35 | 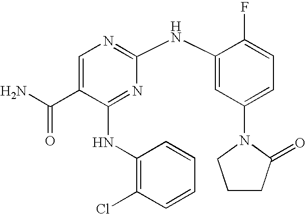 | 4-(2-Chloro-phenylamino)-2- [2-fluoro-5-(2-oxo-pyrrolidin- 1-yl)-phenylamino]- pyrimidine-5-carboxylic acid amide | not det. | 441 |
| 36 | 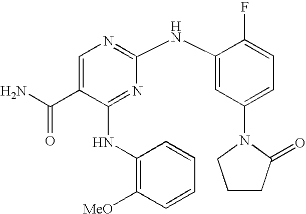 | 2-[2-Fluoro-5-(2-oxo- pyrrolidin-1-yl)-phenylamino]- 4-(2-methoxy-phenylamino)- pyrimidine-5-carboxylic acid amide | B 2.88 | 437 |
| 37 | 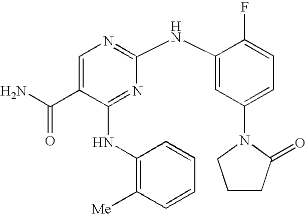 | 2-[2-Fluoro-5-(2-oxo- pyrrolidin-1-yl)-phenylamino]- 4-o-tolylamino-pyrimidine-5- carboxylic acid amide | B 2.95 | 421 |
| 38 | 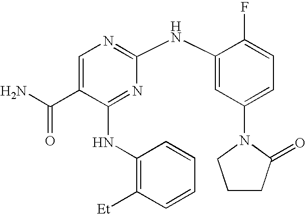 | 4-(2-Ethyl-phenylamino)-2-[2- fluoro-5-(2-oxo-pyrrolidin-1- yl)-phenylamino]-pyrimidine- 5-carboxylic acid amide | B 3.14 | 435 |
| 39 | 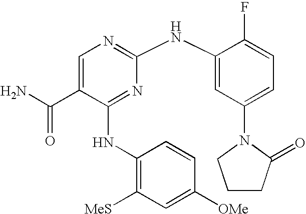 | 2-[2-Fluoro-5-(2-oxo- pyrrolidin-1-yl)-phenylamino]- 4-(4-methoxy-2- methylsulfanyl)-pyrimidine-5- carboxylic acid amide | C 1.92 | 483 |
| 40 | 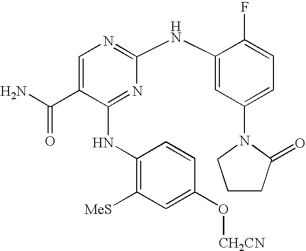 | 4-(4-Cyanomethoxy-2- methylsulfanyl-phenylamino)- 2-[2-fluoro-5-(2-oxo- pyrrolidin-1-yl)-phenylamino]- pyrimidine-5-carboxylic acid amide | E 1.32 | 508 |
| 41 | 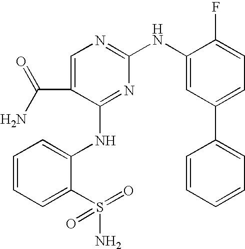 | 2-(4-Fluoro-biphenyl-3-yl- amino)-4-(2-sulfamoyl- phenylamino)-pyrimidine-5- carboxylic acid amide | D 2.76 | 479 |
| 42 | 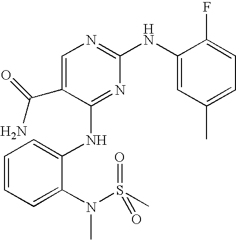 | 2-(2-Fluoro-5-methyl- phenylamino)-4-[2- methanesulfonyl-methyl- amino)-phenylamino]- pyrimidine-5-carboxylic acid amide | D 2.58 | 445 |
| 43 | 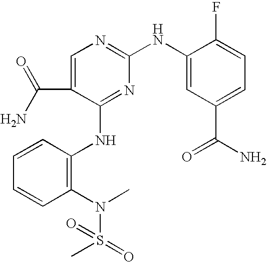 | 2-(5-Carbamoyl-2-fluoro- phenylamino)-4-[2- (methanesulfonyl-methyl- amino)-phenylamino]- pyrimidine-5-carboxylic acid amide | D 1.67 | 474 |
| 44 | 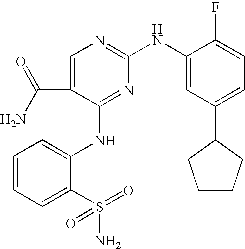 | 2-(5-Cyclopentyl-2-fluoro- phenylamino)-4-(2-sulfamoyl- phenylamino)-pyrimidine-5- carboxylic acid amide | D 2.94 | 471 |
| 45 | 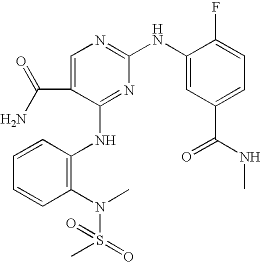 | 2-(2-Fluoro-5- methylcarbamoyl- phenylamino)-4-[2- (methanesulfonyl-methyl- amino)-phenylamino]- pyrimidine-5-carboxylic acid amide | D 1.78 | 488 |
| 46 | 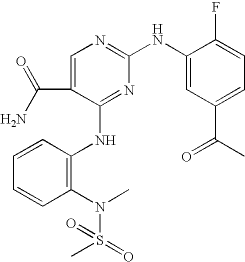 | 2-(5-Acetyl-2-fluoro- phenylamino)-4-[2- metnanesulfonyl-methyl- amino)-phenylamino]- pyrimidine-5-carboxylic acid amide | not det. | 473 |
| 47 | 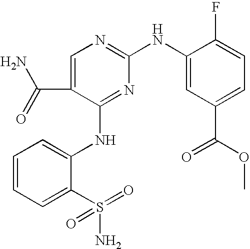 | 3-[5-Carbamoyl-4-(2- sulfamoyl-phenylamino)- pyrimidin-2-ylamino]-4-fluoro- benzoic acid methyl ester | D 2.11 | 461 |
| 48 | 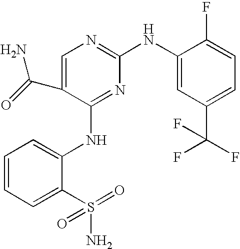 | 2-(2-Fluoro-5-trifluoromethyl- phenylamino)-4-(2-sulfamoyl- phenylamino)-pyrimidine-5- carboxylic acid amide | D 2.56 | 471 |
| 49 | 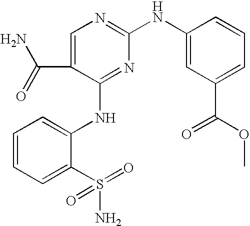 | 3-[5-Carbamoyl-4-(2- sulfamoyl-phenylamino)- pyrimidin-2-ylamino]-benzoic acid methyl ester | D 2.10 | 443 |
| 50 | 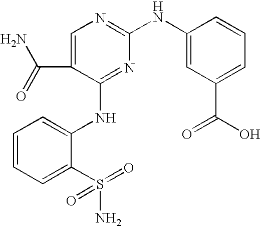 | 3-[5-Carbamoyl-4-(2- sulfamoyl-phenylamino)- pyrimidin-2-ylamino]-benzoic acid | D 1.72 | 429 |
| 51 | 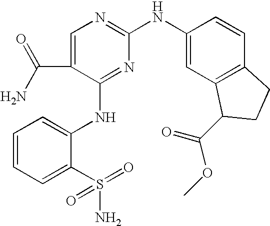 | 6-[5-Carbamoyl-4-(2- sulfamoyl-phenylamino)- pyrimidin-2-ylamino]-indan-1- carboxylic acid methyl ester | D 2.37 | 483 |
| 52 | 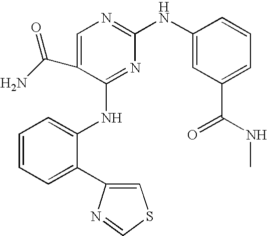 | 2-(2-Fluoro-5- methylcarbamoyl- phenylamino)-4-(2-thiazol-4- yl-phenylamino)-pyrimidine-5- carboxylic acid amide | not det. | 464 |
| 53 | 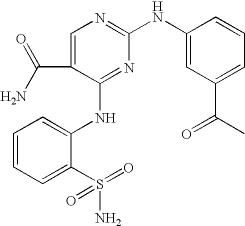 | 2-(3-Acetyl-phenylamino)-4- (2-sulfamoyl-phenylamino)- pyrimidine-5-carboxylic acid amide | D 2.22 | 427 |
| 54 | 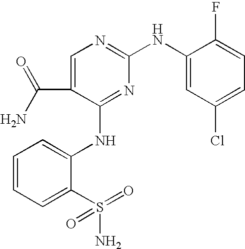 | 2-(5-Chloro-2-fluoro- phenylamino)-4-(2-sulfamoyl- phenylamino)-pyrimidine-5- carboxylic acid amide | D 2.68 | 437 |
| 55 | 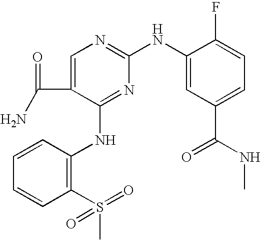 | 2-(2-Fluoro-5- methylcarbamoyl- phenylamino)-4-(2- methanesulfonyl- phenylamino)-pyrimidine-5- carboxylic acid amide | not det. | 459 |
| 56 | 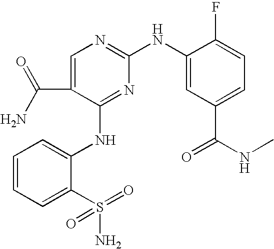 | 2-(2-Fluoro-5- methylcarbamoyl- phenylamino)-4-(2-sulfamoyl- phenylamino)-pyrimidine-5- carboxylic acid amide | D 1.54 | 460 |
| 57 | 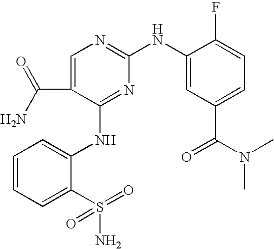 | 2-(5-Dimethylcarbamoyl-2- fluoro-phenylamino)-4-(2- sulfamoyl-phenylamino)- pyrimidine-5-carboxylic acid amide | D 1.68 | 474 |
| 58 | 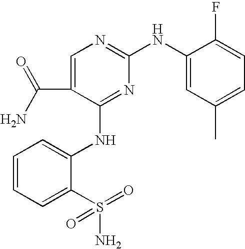 | 2-(2-Fluoro-5-methyl- phenylamino)-4-(2-sulfamoyl- phenylamino)-pyrimidine-5- carboxylic acid amide | D 2.27 | 417 |
| 59 | 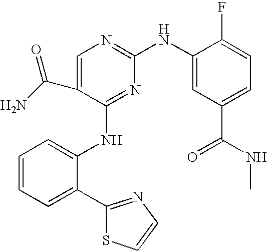 | 2-(2-Fluoro-5- methylcarbamoyl- phenylamino)-4-(2-thiazol-2- yl-phenylamino)-pyrimidine-5- carboxylic acid amide | D 2.09 | 463 |
| 60 | 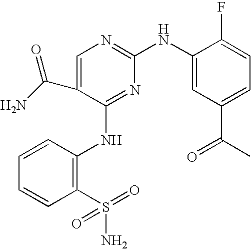 | 2-(5-Acetyl-2-fluoro- phenylamino)-4-(2-sulfamoyl- phenylamino)-pyrimidine-5- carboxylic acid amide | D 1.92 | 445 |
| 61 | 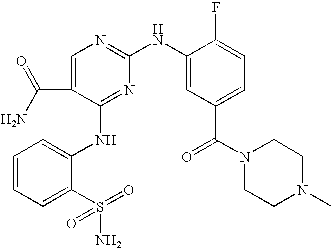 | 2-[2-Fluoro-5-(4-methyl- piperazine-1-carbonyl)- phenylamino]-4-(2-sulfamoyl- phenylamino)-pyrimidine-5- carboxylic acid amide | D 1.24 | 529 |
| 62 | 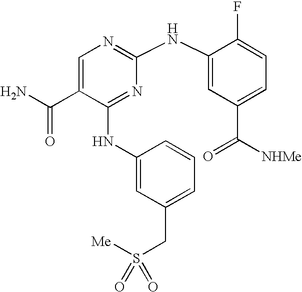 | 2-(2-Fluoro-5- methylcarbamoyl- phenylamino)-4-(3- methanesulfonylmethyl- phenylamino)-pyrimidine-5- carboxylic acid amide | D 1.66 | 473 |
| 63 | 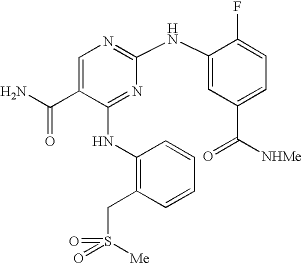 | 2-(2-Fluoro-5- methylcarbamoyl- phenylamino)-4-(2- methanesulfonylmethyl- phenylamino)-pyrimidine-5- carboxylic acid amide | D 1.58 | 473 |
| 64 | 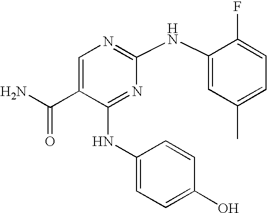 | 2-(2-Fluoro-5-methyl- phenylamino)-4-(4-hydroxy- phenylamino)-pyrimidine-5- carboxylic acid amide | D 2.29 | 354 |
| 65 | 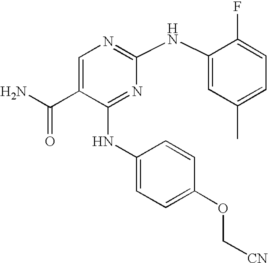 | 4-(4-Cyanomethoxy- phenylamino)-2-(2-fluoro-5- methyl-phenylamino)- pyrimidine-5-carboxylic acid amide | D 2.65 | 392 |
| 66 | 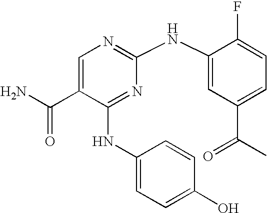 | 2-(5-Acetyl-2-fluoro- phenylamino)-4-(4-hydroxy- phenylamino)-pyrimidine-5- carboxylic acid amide | D 1.94 | 382 |
| 67 | 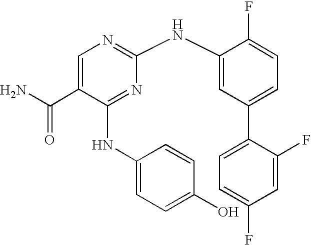 | 4-(4-Hydroxy-phenylamino)- 2-(4,2,4-trifluoro-biphenyl-3- ylamino)-pyrimidine-5- carboxylic acid amide | D 1.88 | 452 |
| 68 | 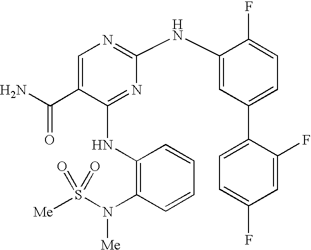 | 4-[2-(Methanesulfonyl- methyl-amino)-phenylamino]- 2-(4,2,4-trifluoro-biphenyl-3- ylamino)-pyrimidine-5- carboxylic acid amide | D 3.14 | 543 |
| 69 | 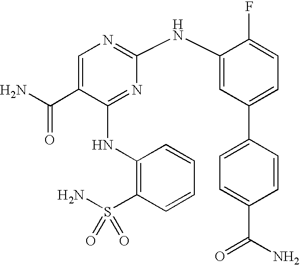 | 2-(4-Carbamoyl-4-fluoro- biphenyl-3-yl-amino)-4-(2- sulfamoyl-phenylamino)- pyrimidine-5-carboxylic acid amine | D 1.99 | 522 |
| 70 | 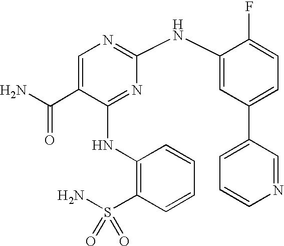 | 2-(2-Fluoro-5-pyridin-3-yl- phenylamino)-4-(2-sulfamoyl- phenylamino)-pyrimidine-5- carboxylic acid amide | G 9.50 | 480 |
| 71 | 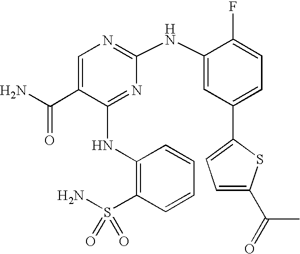 | 2-[5-(5-Acetyl-thiophen-2-yl)- 2-fluoro-phenylamino]-4-(2- sufamoyl-phenylamino)- pyrimidine-5-carboxylic acid amide | G 10.23 | 527 |
| 72 | 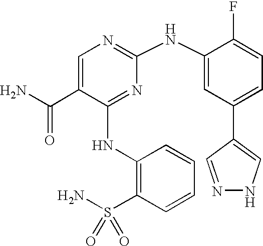 | 2-[2-Fluoro-5-(1H-pyrazol-4- yl)-phenylamino]-4-(2- sulfamoyl-phenylamino)- pyrimidine-5-carboxylic acid amide | D 1.93 | 469 |
| 73 | 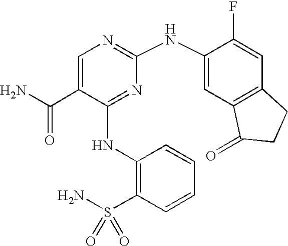 | 2-(6-Fluoro-3-oxo-indan-5-yl- amino)-4-(2-sulfamoyl- phenylamino)-pyrimidine-5- carboxylic acid amide | G 9.20 | 457 |
| 74 | 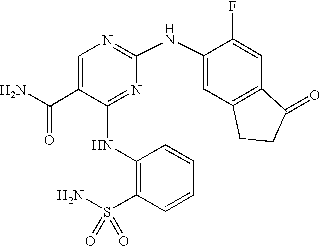 | 2-(6-Fluoro-1-oxo-indan-5-yl- amino)-4-(2-sulfamoyl- phenylamino)-pyrimidine-5- carboxylic acid amide | G 8.93 | 457 |
| 75 | 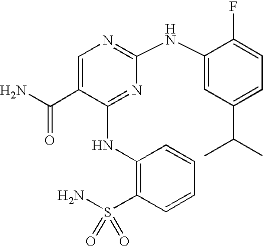 | 2-(2-Fluoro-5-isopropyl- phenylamino)-4-(2-sulfamoyl- phenylamino)-pyrimidine-5- carboxylic acid amide | D 2.68 | 445 |
| 76 | 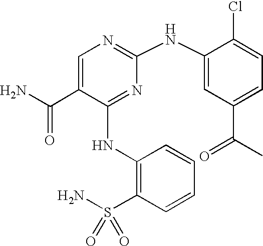 | 2-(5-Acetyl-2-chloro- phenylamino)-4-(2-sulfamoyl- phenylamino)-pyrimidine-5- carboxylic acid amide | D 2.20 | 461 |
| 77 | 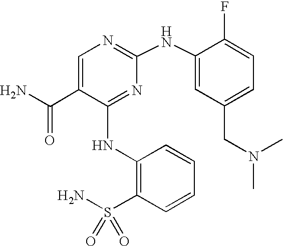 | 2-(5-Dimethylaminomethyl-2- fluoro-phenylamino)-4-(2- sulfamoyl-phenylamino)- pyrimidine-5-carboxylic acid amide | D 1.44 | 460 |
| 78 | 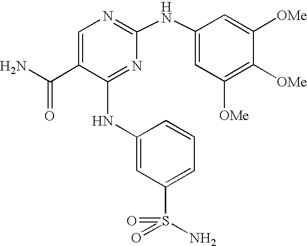 | 4-(3-Sulfamoyl-phenylamino)- 2-(3,4.5-trimethoxy- phenylamino)-pyrimidine-5- carboxylic acid amide | F 7.28 | 475 |
| 79 | 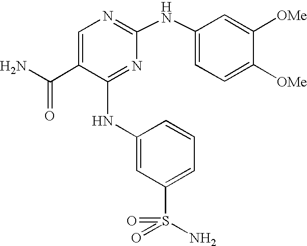 | 2-(3,4-Dimethoxy- phenylamino)-4-(3-sulfamoyl- phenylamino)-pyrimidine-5- carboxylic acid amide | F 7.15 | 445 |
| 80 | 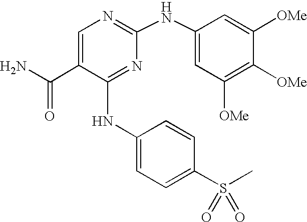 | 4-(4-Methanesulfonyl- phenylamino)-2-(3,4,5- trimethoxy-phenylamino)- pyrimidine-5-carboxylic acid amide | F 7.53 | 474 |
| 81 | 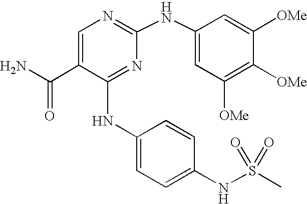 | 4-(4-Methanesulfonylamino- phenylamino)-2-(3,4,5- trimethoxy-phenylamino)- pyrimidine-5-carboxylic acid amide | F 7.35 | 489 |
| 82 | 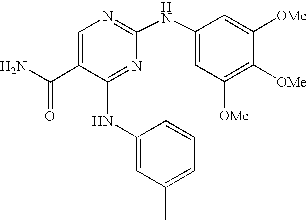 | 4-m-Tolylamino-2-(3,4,5- trimethoxy-phenylamino)- pyrimidine-5-carboxylic acid amide | F 8.68 | 410 |
| 83 | 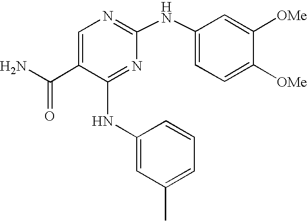 | 2-(3,4-Dimethoxy- phenylamino)-4-m- tolylamino-pyrimidine-5- carboxilic acid amide | F 8.54 | 380 |
| 84 | 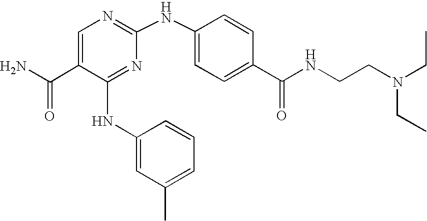 | 2-[4-(2-Diethylamino- ethylcarbamoyl)- phenylamino]-4-m-tolylamino- pyrimidine-5-carboxylic acid amide | F 7.33 | 462 |
| 85 | 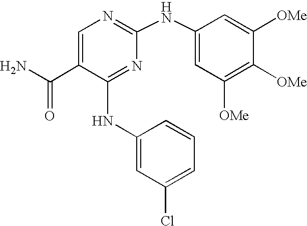 | 4-(3-Chloro-phenylamino)-2- (3,4,5-trimethoxy- phenylamino)-pyrimidine-5- carboxylic acid amide | F 8.74 | 430/432 |
| 86 | 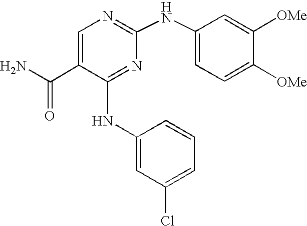 | 4-(3-Chloro-phenylamino)-2- (3,4-dimethoxy- phenylamino)-pyrimidine-5- carboxylic acid amide | F 8.60 | 400/402 |
| 87 | 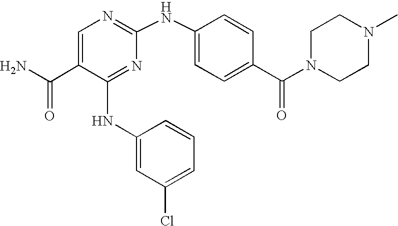 | 4-(3-Chloro-phenylamino)-2- [4-(4-methyl-piperazine-1- carbonyl)-phenylamino]- pyrimidine-5-carboxylic acid amide | F 7.01 | 466/468 |
| 88 | 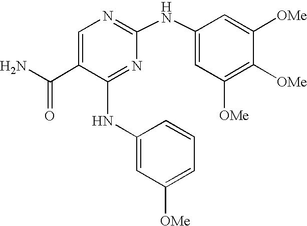 | 4-(3-Methoxy-phenylamino)- 2-(3,4,5-trimethoxy- phenylamino)-pyrimidine-5- carboxylic acid amide | F 8.37 | 426 |
| 89 | 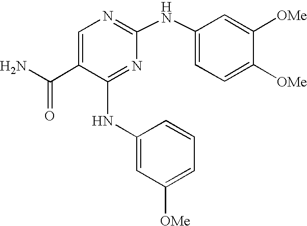 | 2-(3,4-Dimethoxy- phenylamino)-4-(3-methoxy- phenylamino)-pyrimidine-5- carboxylic acid amide | F 8.24 | 396 |
| 90 | 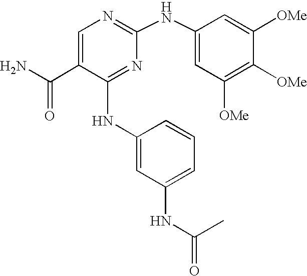 | 4-(3-Acetylamino- phenylamino)-2-(3,4,5- trimethoxy-phenylamino)- pyrimidine-5-carboxylic acid amide | F 7.55 | 453 |
| 91 | 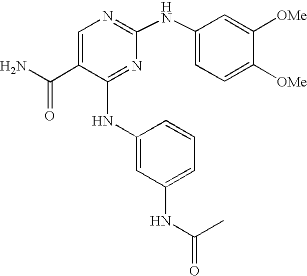 | 4-(3-Acetylamino- phenylamino)-2-(3,4- dimethoxy-phenylamino)- pyrimidine-5-carboxylic acid amide | F 7.21 | 423 |
| 92 | 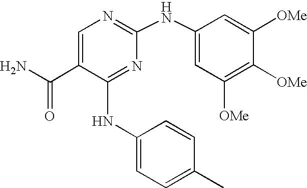 | 4-p-Tolylamino-2-(3,4,5- trimethoxy-phenylamino)- pyrimidine-5-carboxylic acid amide | F 8.67 | 410 |
| 93 | 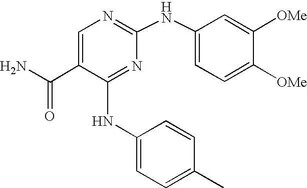 | 2-(3,4-Dimethoxy- phenylamino)-4-p-tolylamino- pyrimidine-5-carboxylic acid amide | F 8.56 | 380 |
| 94 | 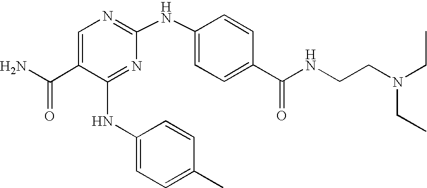 | 2-[4-(2-Diethylamino- ethylcarbamoyl)- phenylamino]-4-p-tolylamino- pyrimidine-5-carboxylic acid amide | F 7.34 | 462 |
| 95 | 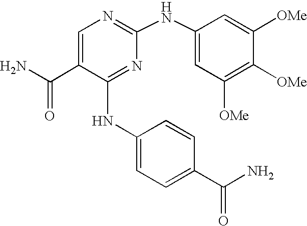 | 4-(4-Carbamoyl- phenylamino)-2-(3,4,5- trimethoxy-phenylamino)- pyrimidine-5-carboxylic acid amide | F 7.02 | 439 |
| 96 | 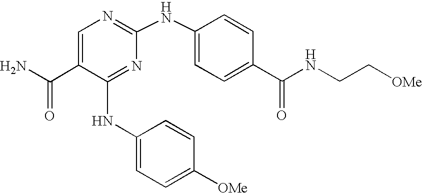 | 2-[4-(2-Methoxy- ethylcarbamoyl)- phenylamino]-4-(4-methoxy- phenylamino)-pyrimidine-5- carboxylic acid amide | F 7.64 | 437 |

The compounds of formula I and their pharmaceutically acceptable salts (“compounds of the invention”), exhibit valuable pharmacological properties when tested in in vitro assays, and are therefore useful as pharmaceuticals.

In particular the compounds of the invention exhibit JAK-3 and JAK-2 kinase inhibiting activities, e.g. as demonstrated in accordance with the following test methods.

In addition, the present compounds have a pronounced selectivity for the above JAK-kinases over other kinases such as for example ZAP-70 or the like.

### 1. JAK Kinase Assays

JAK-2 or JAK-3 enzymatic activity is determined using a time-resolved fluorescence energy transfer technology. The phosphorylation of a synthetic biotinylated peptide substrate (GGEEEYFELVKKKK) by either JAK-2 or JAK-3 in the presence of ATP is quantified using Europium labeled anti phosphotyrosine antibody and Streptavidin-Allophycocyanin Both JAK-2 and JAK-3 enzymes used in these assays contain the kinase domain (JH-1 domain) of the full length proteins and are used as GST fusion proteins.

Inhibitors are dissolved in DMSO. Dilutions are prepared in 90% DMSO followed by additional dilutions steps as required to perform a 8-point concentration-response.

The reaction mix consists of 5 μL of diluted compound, 10 μl of assay buffer and 5 μl of enzyme dilution. After incubation for 60 minutes at room temperature the reaction is stopped by the addition of EDTA. For detection of the product anti-phosphotyrosine antibody and Streptavidin-APC are added and after 60 minutes the samples are measured in an EnVision 2102 Multilabel Reader with excitation wavelength of 320 nm and emission at 665 nm. Alternatively, the kinase assays are performed as described in details by Garcia-Echeverria et al [(2004), Cancer Cell; 5:231-239] in 96-well plates at ambient temperature for 10 min (filter-biding method) or 30 min (flash plates) in a final volume of 30 μL including the following components: GST-JAK-2 or GST-JAK-3, 20 mM Tris-HCl, pH 7.5, 0-1.0 mM MnCl₂, 1-10 mM MgCl₂, 1 mM DTT, 3 μg/mL poly(Glu,Tyr) 4:1, 1% DMSO and 1.0 μM ATP (γ-[³³P]-ATP 0.1 μCi); The assays are terminated by the addition of 20 μl of 125 mM EDTA. The capturing of the phosphorylated peptides by the filter-binding method is performed as following: 40 μL of the reaction mixture are transferred onto Immobilon-PVDF membranes previously soaked for 5 min with methanol, rinsed with water, then soaked for 5 min with 0.5% H₃PO₄ and mounted on vacuum manifold with disconnected vacuum source. After spotting all samples, vacuum is connected and each well rinsed with 200 μl 0.5% H₃PO₄. Free membranes are removed and washed 4× on a shaker with 1.0% H₃PO₄, once with ethanol. Membranes are counted after drying at ambient temperature, mounting in Packard TopCount 96-well frame, and addition of 10 μl/well of Microscint. The plates are eventually sealed and counted in a microplate scintillation counter (TopCount NXT, TopCount NXT HTS, PerkinElmer, Brussels, Belgium).

In these assays, the compounds of the invention have a IC₅₀ value of from 1-1000 nM. For example, compound of Example 6 has an IC₅₀ value of 26 nM in the JAK-3 assay. Compound of Example 5 for example has an IC₅₀ value of 179 nM in the JAK-2 assay.

### 2. JAK-2 In Vivo

The assay may be performed as described by G. Wernig, T. Mercher, R. Okabe, R. L. Levine, B. H. Lee, D. G. Gilliland, Blood First Edition paper, published online Feb. 14, 2006; DOI 10, 1182/blood-2005-12-4824.

### 3. In Vivo Transplantation

Heterotopic heart allotransplantation in the strain combination DA (donor) to Lewis (recipient) is performed according to standard transplantation procedure. Graft function is monitored by daily palpation of the beating donor heart through the abdominal wall. Rejection is considered to be complete when heart beat stops. Prolongation of graft survival is obtained in animals treated with a compound of formula I administered orally at a daily dose of 1 to 100 mg/kg bid.

The compounds of the invention are therefore useful in the prevention or treatment of disorders or diseases where JAK-3 and/or JAK-2 inhibition plays a role, e.g. diseases or disorders mediated by T lymphocytes, B lymphocytes, mast cells and/or eosinophils e.g. acute or chronic rejection of organ or tissue allo- or xenografts, graft-versus-host disease, host-versus-graft disease, atheriosclerosis, vascular occlusion due to vacular injury such as angioplasty, restenosis, fibrosis (especially pulmonary, but also other types of fibrosis, such as renal fibrosis), angiogenesis, hypertension, heart failure, chronic obstructive pulmonary disease, CNS disease such as Alzheimer disease or amyotrophic lateral sclerosis, cancer, infectious disease such as AIDS, septic shock or adult respiratory distress syndrome, ischemia/reperfusion injury e.g. myocardial infarction, stroke, gut ischemia, renal failure or hermorrhage shock, or traumatic shock. The compounds of the invention are also useful in the treatment and/or prevention of acute or chronic inflammatory diseases or disorders or autoimmune diseases e.g. sarcoidosis, fibroid lung, idiopathic interstitial pneumonia, obstructive airways disease, including conditions such as asthma, intrinsic asthma, extrinsic asthma, dust asthma, particularly chronic or inveterate asthma (for example late asthma and airway hyperreponsiveness), bronchitis, including bronchial asthma, infantile asthma, rheumatoid arthritis, osteoarthritis, systemic lupus erythematosus, nephrotic syndrome lupus, Hashimoto's thyroiditis, multiple sclerosis, myasthenia gravis, type I diabetes mellitus and complications associated therewith, type II adult onset diabetes mellitus, uveitis, nephrotic syndrome, steroid dependent and steroid-resistant nephrosis, palmoplantar pustulosis, allergic encephalomyelitis, glomerulonephritis, psoriasis, psoriatic arthritis, atopic eczema (atopic dermatitis), allergic contact dermatitis, irritant contact dermatitis and further eczematous dermatitises, seborrheic dermatitis, lichen planus, pemphigus, bullous pemphigoid, epidermolysis bullosa, urticaria, angioedemas, vasculitides, erythemas, cutaneous eosinophilias, acne, alopecia greata, eosinophilic fasciitis, atherosclerosis, conjunctivitis, keratoconjunctivitis, keratitis, vernal conjunctivitis, uveitis associated with Behcet's disease, herpetic keratitis, conical cornea, Sjoegren's syndrome, dystorphia epithelialis corneae, keratoleukoma, ocular pemphigus, Mooren's ulcer, scleritis, Graves' opthalmopathy, severe intraocular inflammation, inflammation of mucosa or blood vessels such as leukotriene B4-mediated diseases, gastric ulcers, vascular damage caused by ischemic diseases and thrombosis, ischemic bowel disease, inflammatory bowel disease (e.g. Crohn's disease or ulcerative colitis), necrotizing enterocolitis, renal diseases including interstitial nephritis, Goodpasture's syndrome hemolytic uremic syndrome and diabetic nephropathy, nervous diseases selected from multiple myositis, Guillain-Barre syndrome, Meniere's disease and radiculopathy, collagen disease including scleroderma, Wegener's granuloma and Sjogren' syndrome, chronic autoimmune liver diseases including autoimmune hepatitis, primary biliary cirrhosis and sclerosing cholangitis), partial liver resection, acute liver necrosis (e.g. necrosis caused by toxins, viral hepatitis, shock or anoxia), cirrhosis, fulminant hepatitis, pustular psoriasis, Behcet's disease, active chronic hepatitis, Evans syndrome, pollinosis, idiopathic hypoparathyroidism, Addison disease, autoimmune atrophic gastritis, lupoid hepatitis, tubulointerstitial nephritis, membranous nephritis, or rheumatic fever. The compounds of formula I are useful for treating tumors, e.g. breast cancer, genitourinary cancer, lung cancer, gastrointestinal cancer, epidermoid cancer, melanoma, ovarian cancer, pancreas cancer, neuroblastoma, head and/or neck cancer or bladder cancer, or in a broader sense renal, brain or gastric cancer; in particular (i) a breast tumor; an epidermoid tumor, such as an epidermoid head and/or neck tumor or a mouth tumor; a lung tumor, for example a small cell or non-small cell lung tumor; a gastrointestinal tumor, for example, a colorectal tumor; or a genitourinary tumor, for example, a prostate tumor (especially a hormone-refractory prostate tumor); or (ii) a proliferative disease that is refractory to the treatment with other chemothe-rapeutics; or (iii) a tumor that is refractory to treatment with other chemotherapeutics due to multidrug resistance. They are also useful for treating tumors of blood and lymphatic system (e.g. Hodgkin's disease, Non-Hodgkin's lymphoma, Burkitt's lymphoma, AIDS-related lymphomas, malignant immunoproliferative diseases, multiple myeloma and malignant plasma cell neoplasms, lymphoid leukemia, acute or chronic myeloid leukemia, acute or chronic lymphocytic leukemia, monocytic leukemia, other leukemias of specified cell type, leukemia of unspecified cell type, other and unspecified malignant neoplasms of lymphoid, haematopoietic and related tissues, for example diffuse large cell lymphoma, T-cell lymphoma or cutaneous T-cell lymphoma). Myeloid cancer includes e.g. acute or chronic myeloid leukaemia.

Where a tumor, a tumor disease, a carcinoma or a cancer are mentioned, also metastasis in the original organ or tissue and/or in any other location are implied alternatively or in addition, whatever the location of the tumor and/or metastasis.

For the above uses the required dosage will of course vary depending on the mode of administration, the particular condition to be treated and the effect desired. In general, satisfactory results are indicated to be obtained systemically at daily dosages of from about 0.02 to 25 mg/kg per body weight. An indicated daily dosage in the larger mammal, e.g. humans, is in the range from about 0.2 mg to about 2 g, conveniently administered, for example, in divided doses up to four times a day or in retard form. Suitable unit dosage forms for oral administration comprise from ca. 0.1 to 500 mg active ingredient.

The compounds of the invention may be administered by any conventional route, in particular parenterally, for example in the form of injectable solutions or suspensions, enterally, e.g. orally, for example in the form of tablets or capsules, topically, e.g. in the form of lotions, gels, ointments or creams, or in a nasal or a suppository form. Topical administration is e.g. to the skin. A further form of topical administration is to the eye. Pharmaceutical compositions comprising a compound of the invention in association with at least one pharmaceutical acceptable carrier or diluent may be manufactured in conventional manner by mixing with a pharmaceutically acceptable carrier or diluent.

The compounds of formula I may be administered in free form or in pharmaceutically acceptable salt form, e.g. as indicated above. Such salts may be prepared in conventional manner and exhibit the same order of activity as the free compounds.

In accordance with the foregoing, the present invention also provides:

(1) A compound of formula I or a pharmaceutically acceptable salt thereof, for use as a pharmaceutical;
(2) A compound of formula I or a pharmaceutically acceptable salt thereof, for use as a JAK-3 and/or JAK-2 inhibitor, for example for use in any of the particular indications hereinbefore set forth;
(3) A pharmaceutical composition, e.g. for use in any of the indications herein before set forth, comprising a compound of formula I or a pharmaceutically acceptable salt thereof, together with one or more pharmaceutically acceptable diluents or carriers therefor.
(4) A method for the treatment or prevention of a disease or condition in which JAK-3 and/or JAK-2 activation plays a role or is implicated, e.g. for the treatment of any of particular indication hereinbefore set forth in a subject in need thereof which comprises administering to the subject an effective amount of a compound of formula I or a pharmaceutically acceptable salt thereof;
(5) The use of a compound of formula I or a pharmaceutically acceptable salt thereof, for the manufacture of a medicament for the treatment or prevention of a disease or condition in which JAK-3 and/or JAK-2 activation plays a role or is implicated; e.g. as indicated above.

The compounds of the invention may be administered as the sole active ingredient or in conjunction with, e.g. as an adjuvant to, other drugs e.g. in immunosuppressive or immunomodulating regimens or other anti-inflammatory agents, e.g. for the treatment or prevention of allo- or xenograft acute or chronic rejection or inflammatory or autoimmune disorders, a chemotherapeutic agent or an anti-infective agent, e.g. an anti-viral agent such as e.g. an anti-retroviral agent or an antibiotic.

For example, the compounds of the invention may be used in combination with a calcineurin inhibitor, e.g. cyclosporin A, ISA247 or FK 506; a mTOR inhibitor, e.g. rapamycin, 40-O-(2-hydroxyethyO-rapamycin, CCI779, ABT578, TAFA-93, AP23573, AP23464, AP23841, biolimus-7 or biolimus-9; an ascomycin having immuno-suppressive properties, e.g. ABT-281, ASM981, etc.; corticosteroids; cyclophosphamide; azathioprene; methotrexate; leflunomide; mizoribine; mycophenolic acid or salt; mycophenolate mofetil; 15-deoxyspergualine or an immunosuppressive homologue, analogue or derivative thereof; a PKC inhibitor, e.g. as disclosed in WO 02/38561 or WO 03/82859, e.g. the compound of Example 56 or 70; a S1P receptor agonist or modulator, e.g. FTY720 optionally phosphorylated or an analog thereof, e.g. 2-amino-2-[4-(3-benzyloxyphenylthio)-2-chlorophenyl]ethyl-1,3-propanediol optionally phosphorylated or 1-{4-[1-(4-cyclohexyl-3-trifluoromethyl-benzyloxyimino)-ethyl]-2-ethyl-benzyl}-azetidine-3-carboxylic acid or its pharmaceutically acceptable salts; immunosuppressive monoclonal antibodies, e.g., monoclonal antibodies to leukocyte receptors, e.g., MHC, CD2, CD3, CD4, CD7, CDS, CD25, CD28, CD40, CD45, CD52, CD58, CD80, CD86 or their ligands; other immunomodulatory compounds, e.g. a recombinant binding molecule having at least a portion of the extracellular domain of CTLA4 or a mutant thereof, e.g. an at least extracellular portion of CTLA4 or a mutant thereof joined to a non-CTLA4 protein sequence, e.g. CTLA4lg (for ex. designated ATCC 68629) or a mutant thereof, e.g. LEA29Y; adhesion molecule inhibitors, e.g. LFA-1 antagonists, ICAM-1 or -3 antagonists, VCAM-4 antagonists or VLA-4 antagonists, e.g. natalizumab (ANTEGREN®); or antichemokine antibodies or antichemokine receptor antibodies, or low molecular weight chemokine receptor antagonists, e.g. anti MCP-1 antibodies.

A compound of the invention may also be used in combination with other antiproliferative agents. Such antiproliferative agents include, but are not limited to:

(i) aromatase inhibitors, e.g. steroids, especially exemestane and formestane and, in particular, non-steroids, especially aminoglutethimide, vorozole, fadrozole, anastrozole and, very especially, letrozole;
(ii) antiestrogens, e.g. tamoxifen, fulvestrant, raloxifene and raloxifene hydrochloride;
(iii) topoisomerase I inhibitors, e.g. topotecan, irinotecan, 9-nitrocamptothecin and the macromolecular camptothecin conjugate PNU-166148 (compound A1 in WO99/17804);
(iv) topoisomerase II inhibitors, e.g. the antracyclines doxorubicin (including liposomal formulation, e.g. CAELYX™), epirubicin, idarubicin and nemorubicin, the anthraquinones mitoxantrone and losoxantrone, and the podophillotoxines etoposide and teniposide;
(v) microtubule active agents, e.g. the taxanes paclitaxel and docetaxel, the vinca alkaloids, e.g., vinblastine, especially vinblastine sulfate, vincristine especially vincristine sulfate, and vinorelbine, discodermolide and epothilones, such as epothilone B and D;
(vi) alkylating agents, e.g. cyclophosphamide, ifosfamide and melphalan;
(vii) histone deacetylase inhibitors;
(viii) farnesyl transferase inhibitors;
(ix) COX-2 inhibitors, e.g. celecoxib (Celebrex®), rofecoxib (Vioxx®) and lumiracoxib

### (ix) (COX189);

(x) MMP inhibitors;
(xi) mTOR inhibitors;
(xii) antineoplastic antimetabolites, e.g. 5-fluorouracil, tegafur, capecitabine, cladribine, cytarabine, fludarabine phosphate, fluorouridine, gemcitabine, 6-mercaptopurine, hydroxyurea, methotrexate, edatrexate and salts of such compounds, and furthermore ZD 1694 (RALTITREXED™), LY231514 (ALIMTA™), LY264618 (LOMOTREXOL™) and OGT719;
(xiii) platin compounds, e.g. carboplatin, cis-platin and oxaliplatin;
(xiv) compounds decreasing the protein kinase activity and further anti-angiogenic compounds, e.g. (i) compounds which decrease the activity of the Vascular Endothelial Growth Factor (VEGF) (b) the Epidermal Growth Factor (EGF), c-Src, protein kinase C, Platelet-derived Growth Factor (PDGF), Bcr-Abl tyrosine kinase, c-kit, Flt-3 and Insulin-like Growth Factor I Receptor (IGF-IR) and Cyclin-dependent kinases (CDKs); (ii) Imatinib, midostaurin, Iressa™ (ZD1839), CGP 75166, vatalanib, ZD6474, GW2016, CHIR-200131, CEP-7055/CEP-5214, CP-547632 and KRN-633; (iii) thalidomide (THALOMID), celecoxib (Celebrex), SU5416 and ZD6126;
(xv) gonadorelin agonists, e.g. abarelix, goserelin and goserelin acetate;
(xvi) anti-androgens, e.g. bicalutamide (CASODEX™);
(xvii) bengamides;
(xviii) bisphosphonates, e.g. etridonic acid, clodronic acid, tiludronic acid, pamidronic acid, alendronic acid, ibandronic acid, risedronic acid and zoledronic acid;
(xix) antiproliferative antibodies, e.g. trastuzumab (Herceptin™), Trastuzumab-DM1, erlotinib (Tarceva™), bevacizumab (Avastin™), rituximab (Rituxan®), PRO64553 (anti-CD40) and 2C4 Antibody;
(xx) temozolomide (TEMODAL®).

The structure of the active agents identified by code nos., generic or trade names may be taken from the actual edition of the standard compendium “The Merck Index” or from databases, e.g. Patents International (e.g. IMS World Publications).

In accordance with the foregoing the present invention provides in a yet further aspect:

(6) A method as defined above comprising co-administration, e.g. concomitantly or in sequence, of a therapeutically effective amount of a) a compound of formula I or a pharmaceutically acceptable salt thereof, and b) a second drug substance, said second drug substance being for example for use in any of the particular indications hereinbefore set forth.
(7) A combination, e.g. a kit, comprising a therapeutically effective amount of a compound of formula I or a pharmaceutically acceptable salt thereof, and a second drug substance, said second drug substance being for example as disclosed above.

Where a compound of the invention is administered in conjunction with other immunosuppressive/immunomodulatory, anti-inflammatory or antineoplastic agent, e.g. as disclosed above, dosages of the co-administered drug or agent will of course vary depending on the type of co-drug or -agent employed, or the specific drug or agent used, or the condition being treated and so forth.

1. A compound of formula I 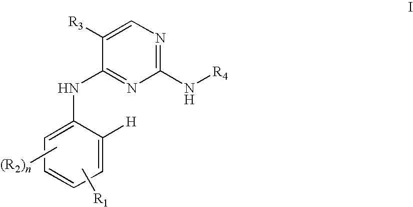
wherein
R₁ and R₂ are independently selected from H; X—SOₘ—Y wherein X is a direct bond, C₁₋₃alkylene, O or NRₐ wherein Rₐ is H or C₁₋₄alkyl; and Y is C₁₋₄alkyl or NR₁₁R₁₂ wherein each of R₁₁ and R₁₂, independently, is H or C₁₋₄alkyl; halogen; OH; C₁₋₇alkyl optionally substituted by OH or C₁₋₆alkoxy; C₁₋₇halogenoalkyl C₁₋₇alkoxy; C₁-C₇alkoxy substituted by cyano; C₁₋₆alkylthio; C₂₋₇alkenyl; C₂₋₇alkenyl; C₃₋₇cycloalkyl; C₃₋₇cycloalkenyl; heterocyclyl; heterocyclylC₁₋₃alkyl; optionally substituted phenyl-R_{b} wherein R_{b} is a direct bond or a bridging group comprising 1 to 4 carbon atoms among which one C atom may be replaced by O or NRₓ, Rₓ being H or C₁₋₃alkyl; optionally substituted heteroaryl-R_{c} wherein R_{c} has independently one of the significances given for R_{b}; heteroaryl N-oxide; or heteroaryl N-oxide C₁₋₃alkyl;
or 2 adjacent R₂ form an annulated 5-12 membered nonaromatic ring optionally containing up to 4 groups selected from CO, NR₁₀, O, S, SO or SO₂;
with the proviso that R₁ and R₂ are not both H;
R₃ is COOH, CONH₂ or CSNH₂;
R₄, is aryl or heteroaryl, each being optionally substituted by 1 to 4 substitutents R₈ selected from halogen; OH; C₁-C₇alkyl optionally substituted by OH or C₁₋₆alkoxy; C₁-C₇alkoxy;
C₁₋₇halogenoalkyl; C₁-C₇alkenyl; C₂₋₇alkynyl; C₃₋₇cycloalkyl; C₃₋₇cycloalkenyl; heterocyclyl; heterocyclylC₁₋₃alkyl; aryl; phenyl; phenyl substituted by C₁-C₇alkyl, C₁₋₆alkoxy, NH₂, NHR₉, NR₉R₉, halogen, C₁₋₃acyl; heteroaryl; C₁₋₃acyl-heteroaryl; heteroarylC₁₋₃alkyl; heteroaryl N-oxideC₀-C₃alkyl; CONH₂; CONHR₉; CONR₉R₈; OC(O)R₉; OC(O)OR₉; OC(O)NHR₉; OC(O)NR₉R₉; OSO₂R₉; COOH; COOR₉; COR₉; X₁COOR₉: CN; NO₂; NH₂; NHR₉; NR₉R₉; X₁NR₉R₉; NHC(O)R₉; NR₉C(O)R₉; NHC(O)NHR₉; NHC(O)NH₂; NR₉C(O)NHR₉; NR₉C(O)NR₉R₉; NHC(O)OR₉; NR₉C(O)OR₉; NHSO₂R₉; N(SO₂R₉)₂; NR₉SO₂R₉; SR₉; S(O)R₃; SO₂R₉; or Si(CH₃)₃;
or 2 adjacent R₈ form an annulated 5-12 membered nonaromatic ring optionally containing up to 4 groups selected from CO, CHCOOH, CHCOOR₉, NR₁₀, O, S, SO or SO₂;
each of R₉, independently, is C₁₋₆alkyl; C₂₋₆alkenyl; C₂₋₆alkynyl; C₂₋₄hydroxyalkyl; R₁₀O—C₂₋₄alkyl; R₁₀R₁₀R₁₀N—C₂₋₄alkyl; C₃₋₆cycloalkyl; cycloalkylC₁₋₃alkyl; phenyl; phenylC₁₋₃alkyl; heteroaryl; heteroarylC₁₋₃alkyl; heterocyclyl; heterocyclylC₁₋₃alkyl;
or 2 R₉ form together with the N atom to which they are attached, a 4 to 7 membered non-aromatic ring optionally containing up to 3 groups selected from CO, NR₁₀, O, S, SO or SO₂;
each of R₁₀, independently, is H; C₁₋₆alkyl; C₂₋₄hydroxyalkyl; or C₃₋₅cycloalkyl;
or 2 R₁₀ form together with the N atom to which they are attached, a 4 to 7 membered non-aromatic ring; and
n is 1 or 2;
m is 1 or 2, preferably 2;
X₁ is a direct bond or C₁₋₆alkylene;
in free form or in sail form.

2. A compound of claim 1, wherein
R₁ is H; X—SOₘ—Y wherein X is a direct bond, C₁₋₃alkylene, O or NRₐ wherein Rₐ is H or C₁₋₄alkyl; and Y is C₁₋₄alkyl or NR₁₁R₁₂ wherein each of R₁₁ and R₁₂) independently, is H or C₁₋₄alkyl;
R₂ is H; halogen; OH; C₁₋₇alkyl optionally substituted by OH or C₁₋₆alkoxy; C₁₋₇halogenoalkyl;
C₁₋₇alkoxy; C₁-C₇alkoxy substituted by cyano; C₁₋₃alkylthio; C₂₋₇alkenyl; C₂₋₇alkynyl;
C₃₋₇cycloalkyl; C₃₋₇cycloalkenyl; heterocyclyl; heterocyclylC₁₋₃alkyl; optionally substituted phenyl-R_{b} wherein R_{b} is a direct bond or a bridging group comprising 1 to 4 carbon atoms among which one C atom may be replaced by O or NRₓ, Rₓ being H or C₁₋₃alkyl; optionally substituted heteroaryl-R_{c} wherein R_{c} has independently one of the significances given for R_{b}; heteroaryl N-oxide; or heteroaryl N-oxide C₁₋₃alkyl;
or 2 adjacent R₂ form an annulated 5-12 membered nonaromatic ring optionally containing up to 4 groups selected from CO, NR₁₀, O, S, SO or SO₂;
with the proviso that R₁, and R₂ are not both H;
R₃ is COOH, CONH₂ or CSNH₂;
R₄, is aryl or heteroaryl, each being optionally substituted by 1 to 4 substitutents R₈selected from halogen; OH; C₁-C₇alkyl optionally substituted by OH or C₁₋₆alkoxy; C₁-C₇alkoxy; C₁₋₇halogenoalkyl; C₂₋₇alkenyl; C₂₋₇alkynyl; C₃₋₇cycloalkyl; C₃₋₇-cycloalkenyl; heterocyclyl; heterocyclylC₁₋₃alkyl; aryl; phenyl; phenyl substituted by C₁-C₇alkyl, C₁₋₆alkoxy, NH₂, NHR₉, NR₉R₉, halogen, C₁₋₃acyl; heteroaryl; C₁₋₃acyl-heteroaryl; heteroarylC₁₋₃alkyl; heteroaryl N-oxideC₀-C₃alkyl; CONH₂; CONHR₉; CONR₉R₉; OC(O)R₉; OC(O)OR₉; OC(O)NHR₉; OC(O)NR₈R₉; OSO₂R₉; COOH; COOR₉; COR₉; X₁COOR₉; CN; NO₂; NH₂; NHR₉; NR₉R₉; X₁NR₉R₉; NHC(O)R₉; NR₉C(O)R₉; NHC(O)NHR₉; NHC(O)NH₂; NR₉C(O)NHR₉; NR₉C(O)NR₉R₉; NHC(O)OR₉; NR₉C(O)OR₉; NHSO₂R₉; N(SO₂R₉)₂; NR₉SO₂R₉; SR₉; S(O)R₉; SO₂R₉; or Si(CH₃)₃;
or 2 adjacent R₉ form an annulated 5-12 membered nonaromatic ring optionally containing up to 4 groups selected from CO, CHCOOH, CHCOOR₉, NR₁₀, O, S, SO or SO₂;
each of R₉, independently, is C₁₋₆alkyl; C₂₋₆alkenyl; C₂₋₆alkynyl; C₂₋₄hydroxyalkyl; R₁₀O—C₂₋₄alkyl; R₁₀R₁₀N—C₂₋₄alkyl; C₃₋₆cycloalkyl; C₁₋₃cycloalkylC₁₋₃alkyl; phenyl; phenylC₁₋₃alkyl; heteroaryl; heteroarylC₁₋₃alkyl; heterocyclyl; heterocyclylC₁₋₃alkyl;
or 2 R₉ form together with the N atom to which they are attached, a 4 to 7 membered non-aromatic ring optionally containing up to 3 groups selected from CO, NR₁₀, O, S, SO or SO₂;
each of R₁₀, independently, is H; C₁₋₆alkyl; C₂₋₄hydroxyalkyl; or C₃₋₆cycloalkyl;
or 2 R₁₀ form together with the N atom to which they are attached, a 4 to 7 membered non-aromatic ring; and
n is 1 or 2;
m is 1 or 2, preferably 2;
X₁ is a direct bond or C₁₋₆alkylene;
in free form or in salt form.

3. A compound of claim 1, wherein R₈ is CONH₂.

4. A compound in accordance with claim 1, wherein Rₜ is X—SOₘ—Y wherein X is a direct bond, C₁₋₃alkylene, O or NRₐ wherein Rₐ is H or C₁₋₄alkyl; and Y is C₁₋₄alkyl or NR₁₁R₁₂ wherein each of R₁₁ and R₁₂, independently, is H or C₁₋₄alkyl; and wherein m is 1 or 2, preferably 2.

5. A compound in accordance with claim 1 wherein R₁ is H; and R₂ is halogen; OH; C₁₋₇alkyl optionally substituted by OH or C₁₋₆alkoxy; C₁₋₇halogenoalkyl; C₁₋₇alkoxy; C₁-C₇alkoxy substituted by cyano; C₁₋₆alkylthio; C₂₋₇alkenyl; C₂₋₇alkynyl; C₃₋₇cycloalkyl; C₃₋₇cycloalkenyl; heterocyclyl; heterocyclylC₁₋₃alkyl; optionally substituted phenyl-R_{b} wherein R_{b} is a direct bond or a bridging group comprising 1 to 4 carbon atoms among which one C atom may be replaced by O or NRₓ, Rₓ being H or C₁₋₃alkyl; optionally substituted heteroaryl-R_{c} wherein R_{c} has independently one of the significances given for R_{b}; heteroaryl M-oxide; or heteroaryl N-oxide C₁₋₃alkyl;
or 2 adjacent R₂ form an annulated 5-12 membered nonaromatic ring optionally containing up to 4 groups selected from GO, NR₁₀, O, S, SO or SO₃ and
n is 1 or 2.

6. A compound of claim 1, wherein R₃ is CONH₂ and R₄ is aryl being optionally substituted by 1 to 4 substituted Rₑ selected from halogen; OH; C₁-C₇alkyl optionally substituted by OH or C₁₋₆alkoxy; C₁-C₇alkoxy; C₁₋₇halogenoalkyl; C₂₋₇alkenyl; C₂₋₇alkynyl; C₃₋₇cycloalkyl; C₃₋₇cycloalkenyl; heterocyclyl; heterocyclylC₁₋₄alkyl; phenyl; phenyl substituted by C₁-C₇alkyl, C₁₋₆alkoxy, NH₂, NHR₉, NR₉R₉, halogen, C₁₋₃acyl; phenyl substituted by 1-3 halogen; phenyl substituted by 1-3 carbamoyl; heteroaryl; C₁₋₃acyl-heteroaryl; heteroarylC₁₋₃alkyl; heteroaryl N-oxideC₀-C₃alkyl; CONH₂; CONHR₉; CONR₉R₉; OC(O)R₈; OC(O)OR₉; OC(O)NHR₉; OC(O)NR₉R₉; OSO₂R₉; COOH; COOR₉; COR₉; X₁COOR₉; CN; NO₂; NH₂; NHR₉; NR₉R₉; X₁NR₉R₉; NHC(O)R₉; NR₉C(O)R₉; NHC(O)NHR₉; NHC(O)NH₂; NR₉C(O)NHR₉; NR₉C(O)NR₉R₉; NHC(O)OR₉; NR₉C(O)OR₉; NHSO₂R₉; N(SO₂R₉)₂; NR₉SO₂R₉; SRS; S(O)R₉; SO₂R₉; or Si(CH₃)₃;
or 2 adjacent R₈ form an annulated 5-12 membered nonaromatic ring optionally containing up to 4 groups selected from CO, CHCOOH, CHCOOR₃(NR₁₀, O, S, SO or SO₂;
each of R₉, independently, is C₁₋₆alkyl; C₂₋₄alkenyl; C₂₋₄alkynyl; C₂₋₆hydroxyalkyl; R₁₀O—C₂₋₄alkyl; R₁₀R₁₀N—C₂₋₄alkyl; C₃₋₆cycloalkyl; C₃₋₆cycloalkylC₁₋₃alkyl; phenyl; phenylC₁₋₃alkyl; heteroaryl; heteroarylC₁₋₃alkyl; heterocyclyl; heterocyclylC₁₋₄alkyl;
or 2 Rₐ form together with the N atom to which they are attached, a 4 to 7 membered non-aromatic ring optionally containing up to 3 groups selected from CO, NR₁₀, O, S, SO or SO₂;
each of R₁₀, independently, is H; C₁₋₆alkyl; C₂₋₄hydroxyalkyl; or C₃₋₆cycloalkyl;
or 2 R₁₀ form together with the N atom to which they are attached, a 4 to 7 membered non-aromatic ring; and
n is 1 or 2.

7. A compound of claim 1, wherein R₃ is CONH₂ and R₄ is a radical of formula Ia wherein the free valence (atom to which it is attached) is indicated by the free bond 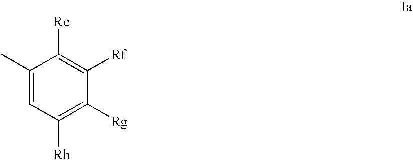
wherein Rₑ is H, Hal, or amino;
R_{f} is H or C₁₋₆alkoxy;
R_{g} is H, C₁₋₆alkoxy, CONHR₉ or CONR₉R₉; and
Rₕ is selected from halogen; C₁-C₇alkyl; C₁₋₆alkoxy; C₁₋₇halogenoalkyl C₃₋₇cycloalkyl; heterocycyl; phenyl; phenyl substituted by C₁-C₇alkyl, C₁₋₆alkoxy, NH₂, NHR₉, NR₉R₉, halogen, C₁₋₃acyl; carbamoylphenyl; heteroaryl; C₁₋₃acyl-heteroaryl; CONH₂; CONHR₉; CONR₉R₉; OC(O)R₉; COOH; COOR₉; COR₅; X₁COOR₃; CN; NO₂; NH₂; NHR₉; NR₉R₉; X₁NR₉R₉; NHC(O)R₉; NR₉C(O)R₉; NHC(O)NHR₉; NHC(O)NH₂; NR₉C(O)NHR₉;
NR₉C(O)NR₉R₉; NHC(O)OR₉; and NR₉C(O)OR₉;
or R_{g} and Rₕ form an annulated 5-12 membered nonaromatic ring optionally containing up to 4 groups selected from CO, CHCOOH, CHCOOR₉, NR₁₀, O, S, SO or SO₂;
wherein R₉, R₁₀, and X₁ are as defined above.

8. A compound in accordance with claim 1, wherein Rₑ is fluoro.

9. A compound of claim 1, wherein R₂. Is hydrogen.

10. A process for the preparation of a compound of formula I as defined in claim 1, comprising converting a compound of formula II 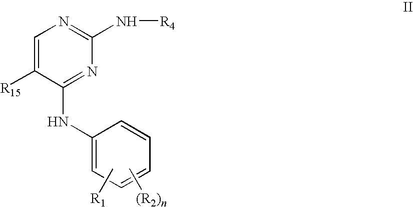
wherein n, R₁(R₂ and R₄ is as defined in claim 1, and R₁₅ is a group which can be converted to R₃,
and recovering the resulting compound of formula I in free or in form of a salt, and, where required, converting the compound of formula I obtained in free form into the desired salt form, or vice versa.

11. A compound of formula I according to claim 1 or a pharmaceutically acceptable salt thereof, for use as a pharmaceutical.

12. A compound of formula I according to claim 1 or a pharmaceutically acceptable salt thereof, for use as a JAK-3 and/or JAK-2 inhibitor.

13. A pharmaceutical composition comprising a compound of formula I according to claim 1 or a pharmaceutically acceptable salt thereof, together with one or more pharmaceutically acceptable diluents or carriers therefor.

14. A method for the treatment or prevention of a disease or condition in which JAK-3 and/or JAK-2 activation plays a role or is implicated, in a subject in need thereof which comprises administering to the subject an effective amount of a compound of formula I according to claim 1 or a pharmaceutically acceptable salt thereof.

15. The use of a compound of formula I according to claim 1 or a pharmaceutically acceptable salt thereof, for the manufacture of a medicament for the treatment or prevention of a disease or condition in which JAK-3 and/or JAK-2 activation plays a role or is implicated.

16. A method according to claim 6 comprising co-administration, e.g. concomitantly or in sequence, of a therapeutically effective amount of a) a compound of formula I as defined in claim 1 or a pharmaceutically acceptable salt thereof, and b) a second drug substance.

17. A combination comprising a therapeutically effective amount of a compound of formula I according to claim 1 or a pharmaceutically acceptable salt thereof, and a second drug substance.
